(19) United States
(12) Patent Application Publication
(10) Pub. No.: US 2025/0179549 A1
(43) Pub. Date: Jun. 5, 2025

(54) Methods of Higher Fidelity RNA Synthesis

(71) Applicant: New England Biolabs, Inc., Ipswich, MA (US)

(72) Inventors: Bijoyita Roy, Medford, MA (US); Tien-Hao Chen, Saugus, MA (US); Vladimir Potapov, Auburndale, MA (US); Jennifer Ong, Salem, MA (US); Nan Dai, Ipswich, MA (US)

(73) Assignee: New England Biolabs, Inc., Ipswich, MA (US)

(21) Appl. No.: 18/845,171

(22) PCT Filed: Apr. 7, 2023
(86) PCT No.: PCT/US2023/065496

§ 371 Date: Sep. 9, 2024

Related U.S. Application Data
(60) Provisional application No. 63/328,654, filed on Apr. 7, 2022.

(51) Int. Cl. C12P 19/34; A61K 38/00; C07K 14/005; C12N 9/12

(52) U.S. Cl. CPC C12P 19/34; C07K 14/005; C12N 9/1252; A61K 38/00; C12Y 207/07006

(57) ABSTRACT

The present disclosure relates, according to some embodiments, to methods and compositions for improving fidelity of nucleotide incorporation during RNA synthesis. Immunogenicity of in vitro transcribed synthetic mRNAs may be reduced and/or therapeutic efficacy may be increased by RNA sequence optimization and/or incorporation of modified uridine analogs (e.g. pseudouridine (Ψ) and N¹-methyl-pseudouridine (m1Ψ)). The present disclosure relates to protocols to improve uridine analog incorporation-without affecting total RNA yield-during IVT. Methods for higher-fidelity incorporation of uridine analogs during IVT include guidelines when choosing ssRNAPs for the generation of modified uridine-containing mRNAs in vitro.

### CROSS-REFERENCE TO RELATED APPLICATIONS

This application is a § 371 application of International Application No. PCT/US2023/065496, filed on Apr. 7, 2023, which claims priority to U.S. Provisional Application No. 63/328,654 filed Apr. 7, 2022, the entire contents of which are hereby incorporated in their entirety by reference.

### SEQUENCE LISTING STATEMENT

This disclosure includes a Sequence Listing submitted electronically in .xml format under the file name “NEB-455.xml” created on Mar. 30, 2023, and having a size of 23.0 KB. This Sequence Listing is incorporated herein in its entirety by this reference.

### BACKGROUND

Synthetic messenger RNAs (mRNAs) have emerged as an attractive modality for vaccines, and they are also being evaluated as a vector for therapeutics. Despite there being several advantages over conventional protein-based approaches, mRNA-based therapeutics are still in early stages of development. Instability of the synthetic mRNAs and the immune responses generated against these synthetic molecules have been key hurdles in the adaptation of this technology, particularly for therapeutic applications where prolonged expression from the synthetic molecule is desirable and repeated dosing of the drug product is required. The use of chemically modified bases in synthetic mRNAs is an innovation that has allowed for both an ameliorated immune response to the synthetic molecules and increased protein expression from the mRNA, thereby providing an unprecedented opportunity to use synthetic mRNAs as a new class of therapeutics for a wide range of indications, including two approved vaccines against COVID-19. It has been shown that the incorporation of pseudouridine (ψ), N¹-methyl-pseudouridine (m1ψ), 5-methylcytosine (m5C), N⁶-methyladenosine (m6A) and 2-thiouridine (s2U) into synthetic mRNAs results in reduced immune responses and increased protein expression in vivo. Pseudouridine-modified mRNAs have been shown to result in reduced activation of 2′-5′-oligoadenylate synthetase (OAS), RNA-dependent protein kinase (PKR), and toll-like receptors. Investigation of w derivatives with improved pharmacological properties led to the identification of m1ψ, the current benchmark for synthetic mRNA-based applications. m1ψ is a naturally occurring modification found in 18S rRNA and tRNAs, and similar to ψ-modified synthetic mRNAs, the presence of m1ψ in synthetic mRNAs has been demonstrated to result in reduced activation of RNA sensors in cells. Furthermore, the presence of m1ψ in synthetic mRNAs show increased translation efficiency in cell-free extracts, multiple mammalian cell lines, and mouse models. The exact mechanism of how m1ψenhances translation is not well understood but it has been demonstrated that presence of m1ψ, alone or in combination with other chemical modifications, can alter ribosome transit time on the modified mRNA, and can increase the mRNA half-life by altering the secondary structure of the synthetic mRNA.

Methods to incorporate modified nucleotides in synthetic mRNAs may include complete substitution of the standard nucleotide with a chemically modified nucleotide during the process of in vitro transcription (IVT) by single-subunit DNA-dependent RNA polymerases (ssRNAPs; such as T7, Hi-T7, T3, KP34, and SP6 RNAP). In contrast to endogenous mRNAs, in which modified nucleotides occur in specific positions in the mRNA, in synthetic mRNAs, the modified nucleotide is present at almost every position where the corresponding naturally occurring nucleobase would be. This complete substitution approach may be preferred by regulatory authorities because it results in less molecule-to-molecule variation. The full and exact implications of incorporating modified nucleotides throughout the body of mRNAs are under investigation. It may be desirable (e.g., for expression/production of the synthetic molecule and/or effectiveness from the drug product) for modified nucleotides to be incorporated in the right place, to be compatible with the functional elements of the mRNA, and to alter few or none of the biological functions of the mRNA. Numerous studies have demonstrated that ssRNAPs can incorporate chemically modified nucleotides into RNA, but it is unclear whether all ss-RNAPs incorporate the modified nucleotides with comparable fidelity.

### SUMMARY

Accordingly, needs have arisen for improved methods and compositions for faithful incorporation of nucleotides during RNA synthesis. The present disclosure relates to systems, apparatus, compositions, and/or methods of synthesizing RNA with improved fidelity. For example, methods and compositions are disclosed herein that take into consideration results-effective variables. A Pacific Biosciences Single Molecule Real-Time (SMRT) sequencing-based assay used to determine the combined transcription and reverse transcription errors revealed that T7 RNAP exhibited higher combined error rates for modified ribonucleotides (e.g., ψ-, m6A- and 5-hydroxymethylcytidine (hm5C)-modified RNAs) than unmodified nucleotides. T7 RNA polymerase displayed increased misincorporation of ψ across from dT templated bases relative to uridine. In light of the desirability of m1ψ for mRNA vaccines, the incorporation of m1ψduring in vitro transcription with ssRNAPs was evaluated. The fidelity with which m1ψ is incorporated during in vitro transcription may be relevant to both the Spike Vax (mRNA-1273) and COMIRNATY® (BNT162b2) SARS-COV2 mRNA vaccines, which are synthesized by substituting uridine with m1ψ throughout the body of the mRNA. The fidelity of m1ψ incorporation with five commonly used ssRNAPs (T7, Hi-T7, T3, KP34 and SP6 RNAPs) was investigated and compared to the fidelity with which uridine and w are incorporated. By using four different RNA sequences, including two functional mRNA sequences, the effects of sequence context in promoting misincorporation of uridine analogs was analyzed and rA→rU substitution errors (i.e., misincorporation of rU at a position in the synthesized RNA where, if faithfully transcribed, rA would have been incorporated instead) were identified as the predominant errors when uridine analogs (m1ψ and w) are present in the reaction. In view of the substitution errors observed, sequence optimization of the synthetic mRNA was combined together with an altered RNA synthesis process to reduce the uridine analog incorporation error during in vitro transcription. The present disclosure provides methods to synthesize mRNAs with improved fidelity of uridine analog incorporation and provide considerations for choosing ssRNAPs for the generation of modified nucleotide-containing mRNAs in vitro.

The present disclosure relates, in some embodiments, to methods comprising contacting a polynucleotide comprising a template sequence; a mixture of ribonucleotide triphosphates (e.g., canonical rNTPs and analogs thereof), wherein the molar ratio of three species of ribonucleotide triphosphates is 1:1:1 and the molar ratio of one species (e.g., rATP) of ribonucleotide triphosphates to any of the other three species (e.g., rGTP, rCTP, and rUTP or rGTP, rCTP, and ψTP or rGTP, rCTP, and m1ψTP) is other than 1:1 (e.g., more than 1:1, more than 2:1, less than 1:1, 1:2 or less than 1:2); and an RNA polymerase, to produce a polyribonucleotide transcription product. A polyribonucleotide transcription product may comprise a sequence complementary to the template sequence, wherein the polyribonucleotide transcription product comprises fewer base substitution errors than a polyribonucleotide transcription product produced by contacting the polynucleotide template, the RNA polymerase, and an equimolar mixture of the same ribonucleotide triphosphates. In some embodiments, the RNA polymerase is T3 RNA polymerase, T7 RNA polymerase, Hi-T7 RNA polymerase, KP34 RNA polymerase, or SP6 RNA polymerase. A polyribonucleotide transcription product may be capped enzymatically or chemically, in some embodiments. For example, a method may include enzymatically capping (e.g., with Faustovirus capping enzyme or a vaccinia capping enzyme) the polyribonucleotide transcription product to produce a capped polyribonucleotide following the contacting. In some embodiments, the contacting may comprise contacting the polynucleotide, the mixture (of rNTPs), the RNA polymerase and a chemical cap analog to produce a chemically capped polyribonucleotide transcription product.

A method, according to some embodiments, may comprise contacting the polyribonucleotide transcription product (or the capped polyribonucleotide transcription product) and one or more pharmaceutically acceptable additives to produce a pharmaceutical dosage form (e.g., an aerosol, an injection solution, a liquid, a tablet). In some embodiments, a method may include contacting the polyribonucleotide transcription product and one or more additives selected from lipidoids, liposomes, polymers, lipoplexes, peptides, proteins, cells transfected with HCMV RNA vaccines, hyaluronidase, and nanoparticles.

Substitution errors associated with a specific base (e.g., uracil) may be addressed by engineering a template sequence to replace nucleosides with that base. For example, redundancy of the genetic code and/or codon usage in an organism may allow some nucleosides to be replaced with another nucleoside without changing the amino acid sequence of an encoded protein. In some embodiments, a polynucleotide comprising a template sequence may encode a polypeptide having an amino acid sequence and the coding sequence may comprise no more than 105% (or no more than 101%, no more than 102%, no more than 103%, no more than 104%, no more than 108%, no more than 110%, no more than 115%, or no more than 120%) the fewest number of uridines possible to encode the amino acid sequence.

The present disclosure relates, according to some embodiments, to methods for reducing base substitution errors in a polyribonucleotide transcription product produced by an RNA polymerase. For example, a method may comprise contacting a polynucleotide having a template sequence; a composition comprising ribonucleotide triphosphates (e.g., canonical rNTPs and optionally analogs thereof), wherein the molar ratio of the ribonucleotide triphosphates (and optional analogs thereof) is proportional (e.g., equal±10%) to the molar ratio of bases in a sequence complementary to the template sequence; and the RNA polymerase, to produce the polyribonucleotide transcription product, wherein the polynucleotide transcription product comprises the complementary sequence and wherein the polyribonucleotide transcription product comprises fewer base substitution errors than a polyribonucleotide transcription product produced by contacting the polynucleotide, the RNA polymerase, and a composition comprising the same ribonucleotide triphosphates but in an equimolar ratio. A method may comprise enzymatically or chemically capping a polyribonucleotide transcription product to produce a capped polyribonucleotide transcription product.

The present disclosure relates, in some embodiments, to methods comprising contacting (a) a polynucleotide comprising a template sequence, wherein the molar ratio of bases in a sequence complementary to the template sequence is wJ: xK: yL: zM, wherein ψ, x, y, and z are each independently positive numbers from 0-50, J is adenosine or an adenosine analog, K is uridine or a uridine analog, L is guanosine or a guanosine analog, and M is cytidine or a cytidine analog; (b) a composition comprising ribonucleotide triphosphates, wherein the molar ratio of the ribonucleotide triphosphates is w′JTP: x′KTP: y′LTP: z′MTP, wherein w′, x′, y′, and z′ are each independently positive numbers from 0-50 (provided no more than 2 of w′, x′, y′, and z′ can equal 0), optionally up to three of w′, x′, y′, and z′ may be equal to one another ±10% (e.g., may be equal to one another), J is adenosine or an adenosine analog, K is uridine or a uridine analog, L is guanosine or a guanosine analog, and M is cytidine or a cytidine analog; and (c) an RNA polymerase (e.g., T3 RNA polymerase, T7 RNA polymerase, Hi-T7 RNA polymerase, KP34 RNA polymerase, or SP6 RNA polymerase), to produce a polyribonucleotide transcription product comprising the sequence complementary to the template sequence. In some embodiments, at least one of w′, x′, y′, and z′ is greater or less than each of the other three of w′, x′, y′, and z′, and the polyribonucleotide transcription product comprises fewer base substitution errors than a polyribonucleotide transcription product produced by contacting the polynucleotide template, the RNA polymerase, and an equimolar mixture of the same ribonucleotide triphosphates. In some embodiments, w′ may be greater (e.g., 1.5× greater) or less than each of x′, y′, and z′. In some embodiments, x′ may be greater or less than (e.g., no more than half of) each of w′, y′, and z′. In some embodiments, y′ may be greater (e.g., 1.5× greater) or less than each of w′, x′, and z′. In some embodiments, z′ may be greater (e.g., 1.5× greater) or less than each of w′, x′, and y′. According to some embodiments, w may equal w′÷10%, x may equal x′±10%, y may equal y′±10%, and/or z may equal z′±10%. At least one of the base substitution errors, according to some embodiments, may be rA→rU. In some embodiments, J may be uridine, pseudouridine, or N¹-methyl-pseudouridine. According to some embodiments, w may be 25-35, x may be 3-15, y may be 25-35, z may be 25-35, w′ may be 25-35, x′ may be 3-15, y′ may be 25-35, and z′ may be 25-35. According to some embodiments (e.g., where J is uridine, pseudouridine, or N¹-methyl-pseudouridine and, optionally, the template is U depleted), w may equal w′±10%, x may equal x′±20%, y may equal y′±10%, and/or z may equal z′±10%. A transcription product may be capped enzymatically (e.g., with Faustovirus capping enzyme or a vaccinia capping enzyme) or chemically, in some embodiments. A method, according to some embodiments, may comprise contacting the transcription product (or a capped transcription product) and one or more pharmaceutically acceptable additives to produce a pharmaceutical dosage form (e.g., an aerosol, an injection solution, a liquid, a tablet). In some embodiments, a method may include contacting the transcription product and one or more additives selected from lipidoids, liposomes, polymers, lipoplexes, peptides, proteins, cells transfected with HCMV RNA vaccines, hyaluronidase, and nanoparticles.

### BRIEF DESCRIPTION OF THE FIGURES

FIGS. 1A, 1B, and 1C show example bioanalyzer traces demonstrating the integrity of a 1707 nucleotide long Cypridina luciferase mRNA synthesized with T7 RNA polymerase. FIG. 1A shows results for reactions containing uridine. FIG. 1B shows results for reactions containing pseudouridine. FIG. 1C shows results for reactions containing N¹-methylpseudouridine. Irrespective of the uridine analog present in the in vitro transcription reaction, full-length RNA of expected size was obtained. “FU”=fluorescence units.

FIG. 2 shows an example UHPLC trace demonstrating the incorporation of uridine, pseudouridine or N¹-methylpseudouridine in a 1707 nucleotide long Cypridina luciferase mRNA when reactions were performed with T7 RNA polymerase. RNA synthesized with SP6 RNA polymerase is shown as a control in this figure.

FIG. 3 shows example incorporation efficiency of uridine, pseudouridine or N¹-methylpseudouridine in a 1707 nucleotide long Cypridina luciferase mRNA when reactions were performed with T7 RNA polymerase. Both pseudouridine and N¹-methylpseudouridine are incorporated efficiently in the full-length RNA.

FIG. 4A shows example combined first strand error rates observed in three different RNA sequences when T7 RNA polymerase was used for in vitro transcription in the presence of uridine, pseudouridine or N¹-methylpseudouridine. RNA 1/5 represents artificial RNA sequences that have been permutated to include every four-base combination. RNA 2 encodes for Cypridina luciferase mRNA and RNA 3 encodes part of the Bnt162b/Comirnaty mRNA. First strand error rates are the combined error rates of T7 RNA polymerase and ProtoscriptII reverse transcriptase. FIG. 4B shows example distribution of substitution, deletion and insertion errors as a percentage of the total error rates. For all three RNA sequences, presence of pseudouridine in the reaction resulted in higher error rates and substitution errors were more prevalent than insertion or deletion errors.

FIG. 5 shows example base substitution error profile observed for uridine-, pseudouridine-, and N¹-methylpseudouridine-containing RNA sequences when in vitro transcription was performed with T7 RNA polymerase. RNA 1/5 represent artificial RNA sequences that has been permutated to include every four-base combination. RNA 2 encodes for Cypridina luciferase mRNA and RNA 3 encodes part of the Bnt162b/Comirnaty mRNA. Base substitution errors observed when in vitro transcription is performed with T7 RNA polymerase are similar, irrespective of the RNA sequence.

FIG. 6A shows an example bioanalyzer trace demonstrating the integrity of a 1707 nucleotide long Cypridina luciferase mRNA synthesized with SP6 RNA polymerase in reactions containing uridine. FIG. 6B shows an example bioanalyzer trace demonstrating the integrity of a 1707 nucleotide long Cypridina luciferase mRNA synthesized with SP6 RNA polymerase in reactions containing pseudouridine. FIG. 6C shows an example bioanalyzer trace demonstrating the integrity of a 1707 nucleotide long Cypridina luciferase mRNA synthesized with SP6 RNA polymerase in reactions containing N¹-methylpseudouridine. Irrespective of the uridine analog present in the in vitro transcription reaction, full-length RNA of expected size was obtained.

FIG. 7 shows an example UHPLC trace demonstrating the incorporation of uridine, pseudouridine or N¹-methylpseudouridine in a 1707 nucleotide long Cypridina luciferase mRNA when reactions were performed with SP6 RNA polymerase. RNA synthesized with T7 RNA polymerase is shown as a control in this figure.

FIG. 8 shows incorporation efficiency of uridine, pseudouridine or N¹-methylpseudouridine in a 1707 nucleotide long Cypridina luciferase mRNA when example reactions were performed with SP6 RNA polymerase. Both pseudouridine and N¹-methylpseudouridine are incorporated efficiently in the full-length RNA.

FIG. 9A shows combined first strand error rates observed in two different RNA sequences when SP6 RNA polymerase was used for example in vitro transcription reactions in the presence of uridine, pseudouridine or N¹-methylpseudouridine. RNA 1/5 represent artificial RNA sequences that has been permutated to include every four-base combination. RNA 2 encodes for Cypridina luciferase mRNA. First strand error rates are the combined error rates of SP6 RNA polymerase and ProtoscriptII reverse transcriptase. FIG. 9B shows example distribution of substitution, deletion and insertion errors as a percentage of the total error rates. For both the RNA sequences, presence of pseudouridine in the reaction resulted in higher error rates and substitution errors were more prevalent than insertion or deletion errors.

FIG. 10 shows example base substitution error profile observed for uridine-, pseudouridine-, and N¹-methylpseudouridine-containing RNA sequences when in vitro transcription was performed with SP6 RNA polymerase. RNA 1/5 represent artificial RNA sequences that has been permutated to include every four-base combination. RNA 2 encodes for Cypridina luciferase mRNA. This figure also demonstrates that the base substitution errors that are observed when in vitro transcription is performed with SP6 RNA polymerase is similar, irrespective of the RNA sequence.

FIGS. 11A-11R each show an example bioanalyzer trace demonstrating the integrity of three different RNA sequences synthesized with T7 RNA polymerase in reactions. FIGS. 11A, 11B, 11C, 11D, 11E, and 11F show results of reactions using RNA7. FIGS. 11G, 11H, 11I, 11J, 11K, and 11L show results of reactions using RNA8. FIGS. 11M, 11N, 110, 11P, 11Q, and 11R show results of reactions using RNA9. FIGS. 11A, 11D, 11G, 11J, 11M, and 11P show results of reactions with uridine. FIGS. 11B, 11E, 11H, 11K, 11N, and 11Q show results of reactions with pseudouridine. FIGS. 11C, 11F, 11I, 11L, 110, and 11R show results of reactions with N¹-methylpseudouridinc. FIGS. 11A, 11B, 11C, 11G, 11H, 11I, 11M, 11N, and 110 show results of reactions with rNTPs in equal molar amounts (“equal”). FIGS. 11D, 11E, 11F, 11J, 11K, 11L, 11P, 11Q, and 11R show results of reactions with rNTPs in molar amounts proportional to the composition of the RNA sequence to be synthesized (“proportional”). Irrespective of the uridine analog present in the in vitro transcription reaction, full-length RNA of expected size was obtained. RNA7 composition: 30.9% A, 33.4% C, 30.2% G, and 5.5% U; RNA8 composition: 29.7% A, 32.0% C, 31.7% G, and 6.6% U; RNA9 composition: 30.2% A, 30.4% C, 27.1% G, and 12.3% U.

FIG. 12 shows comparable RNA yield from example in vitro transcription reactions with T7 RNA polymerase in reactions that contained rNTPs either in equal molar amounts (equal) or in molar amounts proportional to the composition of the RNA sequence to be synthesized (proportional). The reactions were performed with either uridine, pseudouridine or N¹-methylpseudouridine. RNA8 composition: 29.7% A, 32.0% C, 31.7% G, and 6.6% U.

FIG. 13 shows example incorporation efficiency of uridine, pseudouridine or N¹-methylpseudouridine in reactions performed with T7 RNA polymerase with rNTPs either in equal molar amounts (equal) or in molar amounts proportional to the composition of the RNA sequence to be synthesized (proportional). Both pseudouridine and N¹-methylpseudouridine are incorporated efficiently in the full-length RNA. RNA8 composition: 29.7% A, 32.0% C, 31.7% G, and 6.6% U.

FIGS. 14A, 14B, 14C, 14D, 14E, and 14F show results for example in vitro transcription reactions. FIGS. 14A, 14C, and 14E show combined first strand error rates observed in three different RNA sequences when T7 RNA polymerase used for in vitro transcription and the reactions contained rNTPs either in equal molar amounts (equal) or in molar amounts proportional to the composition of the RNA sequence to be synthesized (proportional). The reactions were performed with either uridine, pseudouridine or N¹-methylpseudouridine. First strand error rates are the combined error rates of T7 RNA polymerase and ProtoscriptII reverse transcriptase.

FIG. 14A shows results with RNA7 having the following base composition: 30.9% A, 33.4% C, 30.2% G, and 5.5% U; FIG. 14C shows results with RNA8 having the following base composition: 29.7% A, 32.0% C, 31.7% G, and 6.6% U; FIG. 14E shows results with RNA9 having the following base composition: 30.2% A, 30.4% C, 27.1% G, and 12.3% U.

FIGS. 14B, 14D, and 14F show distribution of substitution, deletion and insertion errors as percentage of the total error rates using the sequences of FIGS. 14A, 14C, and 14E, respectively. For all three RNA sequences, the combined total error was reduced when the molar ratio of rNTPs were proportional to the nucleotide composition of the RNA sequence to be synthesized.

FIG. 15 shows reduction in base substitution error profile observed for uridine, pseudouridine-, and N¹-methylpseudouridine-containing RNA sequences when T7 RNA polymerase was used for in vitro transcription and the molar ratio of rNTPs was proportional to the composition of the RNA sequence to be synthesized (proportional). First strand error rates are the combined error rates of T7 RNA polymerase and ProtoscriptII reverse transcriptase. FIG. 15A shows results with RNA7 having the following base composition: 30.9% A, 33.4% C, 30.2% G, and 5.5% U; FIG. 15B shows results with RNA8 having the following base composition: 29.7% A, 32.0% C, 31.7% G, and 6.6% U; FIG. 15C shows results with RNA9 having the following base composition: 30.2% A, 30.4% C, 27.1% G, and 12.3% U.

FIGS. 16A-16F each show an example bioanalyzer trace demonstrating the integrity of the RNA synthesized with SP6 RNA polymerase. FIG. 16A shows results for uridine reactions with rNTPs in equal molar amounts (“equal”). FIG. 16B shows results for pseudouridine reactions with rNTPs in molar amounts proportional to the composition of the RNA sequence to be synthesized (“proportional”). FIG. 16C shows results for N¹-methylpseudouridine reactions with rNTPs in equal molar amounts (“equal”). FIG. 16D shows results for uridine reactions with rNTPs in molar amounts proportional to the composition of the RNA sequence to be synthesized (“proportional”). FIG. 16E shows results for pseudouridine reactions with rNTPs in equal molar amounts (“equal”). FIG. 16F shows results for N¹-methylpseudouridine reactions with rNTPs in molar amounts proportional to the composition of the RNA sequence to be synthesized (“proportional”). Irrespective of the uridine analog present in the in vitro transcription reaction, full-length RNA of expected size was obtained. RNA7 composition: 30.9% A, 33.4% C, 30.2% G, and 5.5% U.

FIG. 17 shows comparable RNA yield from example in vitro transcription reactions with SP6 RNA polymerase in reactions that contained rNTPs either in equal molar amounts (equal) or in molar amounts proportional to the composition of the RNA sequence to be synthesized (proportional). The reactions were performed with either uridine, pseudouridine or N¹-methylpseudouridine. RNA7 composition: 30.9% A, 33.4% C, 30.2% G, and 5.5% U.

FIG. 18 shows example incorporation efficiency of uridine, pseudouridine or N¹-methylpseudouridine in reactions performed with SP6 RNA polymerase with rNTPs either in equal molar amounts (equal) or in molar amounts proportional to the composition of the RNA sequence to be synthesized (proportional). Both pseudouridine and N¹-methylpseudouridine are incorporated efficiently in the full-length RNA. RNA7 composition: 30.9% A, 33.4% C, 30.2% G, and 5.5% U.

FIG. 19A shows combined first strand error rates observed when SP6 RNA polymerase was used for example in vitro transcription and the reactions contained rNTPs either in equal molar amounts (equal) or in molar amounts proportional to the composition of the RNA sequence to be synthesized (proportional). The reactions were performed with either uridine, pseudouridine or N¹-methylpseudouridine. First strand error rates are the combined error rates of SP6 RNA polymerase and ProtoscriptII reverse transcriptase. FIG. 19B shows example distribution of substitution, deletion and insertion errors as percentage of the total error rates. The combined total error was reduced when rNTPs were proportioned to with the nucleotide composition of the RNA sequence to be synthesized. RNA7 composition: 30.9% A, 33.4% C, 30.2% G, and 5.5% U.

FIG. 20 shows example reduction in base substitution error profile observed for uridine-, pseudouridine-, and N¹-methylpseudouridine-containing RNA sequence when SP6 RNA polymerase was used for in vitro transcription and the rNTPs were included in molar amounts proportional to the composition of the RNA sequence to be synthesized (proportional). First strand error rates are the combined error rates of SP6 RNA polymerase and ProtoscriptII reverse transcriptase. RNA7 composition: 30.9% A, 33.4% C, 30.2% G, and 5.5% U.

FIG. 21A shows combined first strand error rates observed when T7 RNA polymerase was used for example in vitro transcription and the reactions contained excess of rATPs as compared to the other rNTPs. The reactions were performed with either uridine, pseudouridine or N¹-methylpseudouridine. First strand error rates are the combined error rates of T7 RNA polymerase and ProtoscriptII reverse transcriptase. FIG. 21B shows example distribution of substitution, deletion and insertion errors as percentage of the total error rates. The combined total error was reduced when excess of rATP was present in the reaction.

FIG. 22 shows example reduction in base substitution error profile observed for pseudouridine-, and N¹-methylpseudouridine-containing RNA sequence observed when T7 RNA polymerase was used for in vitro transcription and the reactions contained excess of rATPs as compared to the other rNTPs.

FIG. 23A shows combined first strand error rates observed when T7/T3 or SP6 RNA polymerases were used for example in vitro transcription. First strand error rates are the combined error rates of the RNA polymerase and ProtoscriptII reverse transcriptase. FIG. 23B shows distribution of substitution, deletion and insertion errors as percentage of the total error rates.

FIG. 24A shows combined first strand error rates observed from example in vitro transcription reactions performed with T7 RNA polymerase under varying total rNTP concentrations. First strand error rates are the combined error rates of the RNA polymerase and ProtoscriptII reverse transcriptase. FIG. 24B shows example distribution of substitution, deletion and insertion errors as percentage of the total error rates. No difference in total error was observed when the total rNTP concentrations were varied in the reaction.

FIGS. 25A-25I show the sequence context surrounding the rA→rU/dT→dA substitution errors observed when example reactions were performed with T7 RNA polymerase. FIGS. 25A, 25D, and 25G show sequence context for uridine reactions. FIGS. 25B, 25E, and 25H show sequence context for pseudouridine reactions. FIGS. 25C, 25F, and 25I show sequence context for N¹-methylpseudouridine reactions. FIGS. 25A, 25B, and 25C show results with artificial RNA sequences (“RNA 1/5”) that have been permutated to include every four-base combination. FIGS. 25D, 25E, and 25F show results with a luciferase mRNA sequence. FIGS. 25G, 25H, and 25I show results with a Comirnaty mRNA sequence.

FIG. 26A shows combined example first strand error rates observed in RNA 1/5 sequences when KP34 RNA polymerase was used for in vitro transcription in the presence of uridine, pseudouridine or N¹-methylpseudouridine. RNA 1/5 represent artificial RNA sequences that has been permutated to include every four-base combination. First strand error rates are the combined error rates of KP34 RNA polymerase and Protoscript II reverse transcriptase. FIG. 26B shows example distribution of substitution, deletion and insertion errors as percentage of the total error rates. The error rates were comparable when RNA sequences were incorporated with and without modified nucleotides, and substitution errors were predominant.

FIG. 27 shows example base substitution error profile observed for uridine-, pseudouridine-, and N¹-methylpseudouridine-containing RNA 1/5 when in vitro transcription was performed with KP34 RNA polymerase. RNA 1/5 represent artificial RNA sequences that has been permutated to include every four-base combination. This figure shows different substitution profiles when different uridine analogs were incorporated into the transcripts.

FIG. 28A shows example combined first strand error rates observed in RNA 1/5 sequences when Hi-T7 RNA polymerase was used for in vitro transcription at 37° C., 48° C. and 50° C. in the presence of uridine, pseudouridine or N¹-methylpseudouridine. RNA 1/5 represent artificial RNA sequences that has been permutated to include every four-base combination. First strand error rates are the combined error rates of Hi-T7 RNA polymerase and Protoscript II reverse transcriptase. FIG. 28B shows example distribution of substitution, deletion and insertion errors are shown as percentage of the total error rates. For all the three temperatures, the presence of pseudouridine resulted in higher error rates and substitution errors were predominant.

FIG. 29 shows example base substitution error profile observed for uridine-, pseudouridine-, and N/-methylpseudouridine-containing RNA 1/5 sequences when in vitro transcription was performed with Hi-T7 RNA polymerase at 37° C., 48° C. and 50° C. RNA 1/5 represent artificial RNA sequences that has been permutated to include every four-base combination. This figure also demonstrates that the base substitution errors that are observed when in vitro transcription is performed with Hi-T7 RNA polymerase are similar, irrespective of the reaction temperature.

### DETAILED DESCRIPTION

The present disclosure relates, in some embodiments, to faithful incorporation of nucleotides during RNA synthesis. For example, the present disclosure relates to systems, apparatus, compositions, and/or methods of synthesizing RNA with improved fidelity. While it may seem easy or pragmatic to synthesize RNA from a template using equimolar amounts of rNTPs, data disclosed herein reveal that misincorporation errors occur under such conditions. For example, rA→rU/dT→dA substitution errors may be observed when synthesizing RNA from U-depleted templates using equimolar amounts of rNTPs. These errors may be even more frequent when the rNTP mixture includes triphosphates of modified nucleotides, for example, pseudouridine or N¹-methylpseudouridine instead of UTP. To improve fidelity of RNA synthesis, the present disclosure provides, in some embodiments, methods and compositions with ratios of rNTPs other than equimolar ratios (other than, for example, molar ratios of 1:1:1:1).

### General Considerations

Aspects of the present disclosure can be further understood in light of the embodiments, section headings, figures, descriptions and examples, none of which should be construed as limiting the entire scope of the present disclosure in any way. Accordingly, the innovations set forth herein should be construed in view of the full breadth and spirit of the disclosure.

Each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible. Unless otherwise expressly stated to be required herein, each component, feature, and method step disclosed herein is optional and the disclosure contemplates embodiments in which each optional element may be expressly excluded. As such, this statement is intended to serve as antecedent basis for use of such exclusive terminology as “solely,” “only” and the like in connection with the recitation of claim elements or use of a “negative” limitation.

Unless otherwise defined, all technical and scientific terms used herein have the same meaning as commonly understood by one of ordinary skill in the art to which this disclosure belongs. Still, certain terms are defined herein with respect to embodiments of the disclosure and for the sake of clarity and ease of reference.

Sources of commonly understood terms and symbols may include: standard treatises and texts such as Kornberg and Baker, DNA Replication, Second Edition (W. H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); Singleton, et al., Dictionary of Microbiology and Molecular biology, 2d ed., John Wiley and Sons, New York (1994), and Hale & Markham, the Harper Collins Dictionary of Biology, Harper Perennial, N.Y. (1991) and the like.

Numeric ranges are inclusive of the numbers defining the range. All numbers should be understood to encompass the midpoint of the integer above and below the integer i.e., the number 2 encompasses 1.5-2.5. The number 2.5 encompasses 2.45-2.55 etc. When sample numerical values are provided, each alone may represent an intermediate value in a range of values and together may represent the extremes of a range unless specified.

As used herein and in the appended claims, the singular forms “a” and “an” include plural referents unless the context clearly dictates otherwise. For example, the term “a protein” refers to one or more proteins, i.e., a single protein and multiple proteins.

In the context of the present disclosure, “adenosine analog” refers to modified adenosine nucleosides including N⁶-2′-O-dimethyladenosine, N⁶-methyladenine, N¹-methyladenine, and N⁶-acetyladenosine.

In the context of the present disclosure, “buffer” and “buffering agent” refer to a chemical entity or composition that itself resists and, when present in a solution, allows such solution to resist changes in pH when such solution is contacted with a chemical entity or composition having a higher or lower pH (e.g., an acid or alkali). Examples of suitable non-naturally occurring buffering agents that may be used in disclosed compositions, kits, and methods include, for example, Tris, HEPES, TAPS, MOPS, tricine, or MES.

In the context of the present disclosure, “cap” refers to a natural cap, such as ⁷ mG, and to a compound of the general formula R3p₃N¹-[p-N](x), where R3 is a guanine, adenine, cytosine, uridine or analogs thereof (e.g., N⁷-methylguanosine; m7G), p₃ is a triphosphate linkage, N¹ and Nx are ribonucleosides, x is 0-8 and p is, independently for each position, a phosphate group, a phosphorothioate, a phosphorodithioate, an alkylphosphonate, an arylphosphonate, or a N-phosphoramidate linkage. R3 may have an added label at the 2′ or 3′ position of the ribose, and, in some embodiments, the label may be an oligonucleotide, a detectable label such as a fluorophore, or a capture moiety such as biotin or desthiobiotin, where the label may be optionally linked to the ribose of the nucleotide by a linker, for example. Scc, e.g., WO 2015/085142. A cap may have a cap 0 structure, a cap 1 structure or a cap 2 structure (e.g., as reviewed in Ramanathan, Nucleic Acids Res. 2016 44:7511-7526), depending on which enzymes and/or whether SAM is present in the capping reaction.

Caps include dinucleotide cap analogs, e.g., of formula m7G (5′) p₃ (5′) G, in which a guanine nucleotide (G) is linked via its 5′OH to the triphosphate bridge. In some dinucleotide caps the 3′—OH group is replaced with hydrogen or OCH₃ (U.S. Pat. No. 7,074,596; Kore, Nucleosides, Nucleotides, and Nucleic Acids, 2006, 25:15 307-14; and Kore, Nucleosides, Nucleotides, and Nucleic Acids, 2006, 25:337-40). Dinucleotide caps include m7G (5′) p₃G, 3′-OMe-m7G (5′) p₃G (ARCA). Caps also include trinucleotide cap analogs (defined below) as well as other, longer, molecules (e.g., cap that have four, five or six or more nucleotides joined to the triphosphate bridge). In a cap analog, the 2′ and 3′ groups on the ribose of the m7G may be independently selected O-alkyl (e.g., O-methyl), halogen, a linker, hydrogen or a hydroxyl and the sugars 20 in N1 and NX may be independently selected from ribose, deoxyribose, 2′-O-alkyl, 2′-O-methoxyethyl, 2′-O-allyl, 2′-O-alkylamine, 2′-fluororibose, and 2′-deoxyribose. N1 and NX may independently (for each position) comprise a base selected from adenine, uridine, guanine, or cytidine or analogs of adenine, uridine, guanine, or cytidine, and nucleotide modifications can be selected from N⁶-methyladenine, N¹-methyladenine, N⁶-2′-Odimethyladenosine, pseudouridine, N¹-methylpseudouridine, 5-iodouridine, 4-thiouridine, 2-thiouridine, 5-methyluridine, pseudoisocytosine, 5-methoxycytosine, 2-thiocytosine, 5-hydroxycytosine, N⁴-methylcytosine, 5-hydroxymethylcytosine, hypoxanthine, N¹-methylguanine, 06-methylguanine, 1-methyl-guanosine, N²-methylguanosine, N²,N²-dimethyl-guanosine, 2-methyl-2′-O-methyl-guanosine, N²,N²-dimethyl-2′-O-methyl-guanosine, 1-methyl-2′-O-methyl-guanosine, N²,N⁷-dimethyl-2′-O-methyl-guanosine, and isoguanineadenine.

In the context of the present disclosure, “capping” refers to the addition of a cap onto the 5′ end of an RNA. Caps may be added at the 5′ end of an RNA (e.g., an uncapped RNA transcript) chemically or enzymatically apart from transcription or co-transcriptionally to yield a 5′ capped RNA. Capping may or may not be reversible.

In the context of the present disclosure, “cytidine analog” refers to modified cytidine nucleosides including 5-hydroxymethylcytidine, 5-methylcytidine, N⁴-acetylcytidine, 2-thiocytidine, 5-formylcytidine, 2′-O-methylcytidine, N⁴-methylcytidine, and 2′-O-methylcytidine.

In the context of the present disclosure, “Faustovirus capping enzyme” and “FCE” refer to a single-chain enzyme having the RNA capping activity and having the amino acid sequence of positions 1 to 878 of SEQ ID NO: 1 disclosed by U.S. Pat. No. 11,028,379.

In the context of the present disclosure, “guanosine analog” refers to modified adenosine nucleosides including 1-methyl-guanosine, N¹-methyl-guanosine, N²-methylguanosine, N²,N²-dimethyl-guanosine, 2-methyl-2′-O-methyl-guanosine, N²,N²-dimethyl-2′-O-methyl-guanosine, 1-methyl-2′-O-methyl-guanosine, N²,N⁷-dimethyl-2′-O-methyl-guanosine.

In the context of the present disclosure, “in vitro transcription” (IVT) refers to a cell-free reaction in which a DNA template is copied by a DNA-directed RNA polymerase (typically a bacteriophage polymerase) to produce a product that comprises one or more RNA molecules that have been copied from the template.

In the context of the present disclosure, “misincorporation”, with reference to transcription of a template sequence by an RNA polymerase, refers to incorporation of a nucleotide into the nascent strand, where the incorporated nucleotide has a mismatched base (e.g., a base that does not follow Watson-Crick pairing) relative to the base at the corresponding position in the template. Examples of mismatched bases include A-G, A-C, U-G, U-C, G-A, G-T, C-A, C-T, wherein the first letter denotes the base of the nascent RNA strand and the second letter denotes the base of the template (RNA or DNA).

In the context of the present disclosure, “modified nucleoside” refers to nucleosides having a modification on the sugars (e.g., 2′-fluororibose, ribose, 2′-deoxyribose, arabinose, and hexose); and/or in the nucleotide base (e.g., as described in U.S. Pat. No. 8,383,340; WO 2013/151666; U.S. Pat. No. 9,428,535 B2; US 2016/0032316). Modified nucleosides include adenosine analogs, uridine analogs, guanosine analogs, and cytidine analogs.

In the context of the present disclosure, “modified nucleotide” refers to nucleotides having a modification on the sugars (e.g., 2′-fluororibose, ribose, 2′-deoxyribose, arabinose, and hexose); and/or in the phosphate groups (e.g., phosphorothioates and 5′-N-phosphoramidite linkages); and/or in the nucleotide base (e.g., as described in U.S. Pat. No. 8,383,340; WO 2013/151666; U.S. Pat. No. 9,428,535 B2; US 2016/0032316).

In the context of the present disclosure, “non-naturally occurring” refers to a polynucleotide, polypeptide, carbohydrate, lipid, or composition that does not exist in nature. Such a polynucleotide, polypeptide, carbohydrate, lipid, or composition may differ from naturally occurring polynucleotides polypeptides, carbohydrates, lipids, or compositions in one or more respects. For example, a polymer (e.g., a polynucleotide, polypeptide, or carbohydrate) may differ in the kind and arrangement of the component building blocks (e.g., nucleotide sequence, amino acid sequence, or sugar molecules). A polymer may differ from a naturally occurring polymer with respect to the molecule(s) to which it is linked. For example, a “non-naturally occurring” protein may differ from naturally occurring proteins in its secondary, tertiary, or quaternary structure, by having a chemical bond (e.g., a covalent bond including a peptide bond, a phosphate bond, a disulfide bond, an ester bond, and ether bond, and others) to a polypeptide (e.g., a fusion protein), a lipid, a carbohydrate, or any other molecule. Similarly, a “non-naturally occurring” polynucleotide or nucleic acid may contain one or more other modifications (e.g., an added label or other moiety) to the 5′-end, the 3′ end, and/or between the 5′- and 3′-ends (e.g., methylation) of the nucleic acid. A “non-naturally occurring” composition may differ from naturally occurring compositions in one or more of the following respects: (a) having components that are not combined in nature, (b) having components in concentrations not found in nature, (c) omitting one or components otherwise found in naturally occurring compositions, (d) having a form not found in nature, e.g., dried, freeze dried, crystalline, aqueous, and (e) having one or more additional components beyond those found in nature (e.g., buffering agents, a detergent, a dye, a solvent or a preservative).

In the context of the present disclosure, a “pharmaceutical dosage form” refers to a composition having any pharmaceutically acceptable form including, for example, pharmaceutical dosage form listed under the U.S. FDA's NCI concept code for pharmaceutical dosage form C42636.

In the context of the present disclosure, “RNA polymerase” refers to a single-subunit DNA-dependent enzyme that synthesizes a polyribonucleotide from rNTPs with a template. Examples of RNA polymerases include T3 RNA polymerase, T7 RNA polymerase, SP6 polymerase, among others and variants thereof including thermostable variants (e.g., RNA polymerases described in International Application No. PCT/US2017/013179 and U.S. application Ser. No. 15/594,090 (“Hi-T7 RNA polymerase”)).

In the context of the present disclosure, “pharmaceutically acceptable additive” refers to binders, buffers, coatings, carriers, colors, controlled release agents, delivery agents (e.g., liposomes, propellants), diluents, disintegrants, dyes, excipients, diluents, excipients, fillers, lipids, lubricants, salts, sorbants, stabilizers, and/or other agents. Additives including carriers may comprise, for example, fluids, solvents, dispersion media, wetting agents, crowding agents, micelles, lipidoids, liposomes, polymers, lipoplexes, peptides, proteins, salts, surface active agents, isotonic agents, thickeners, emulsifiers, preservatives, stabilizers, solubilizers, buffers, sugars, starches, cellulose, waxes, glycols, polyols, polyesters, polycarbonates, polyanhydrides, hyaluronidase, nanoparticles (e.g., lipid nanoparticles, core-shell nanoparticles, and/or nanoparticle mimics), and combinations thereof. In some embodiments, pharmaceutically acceptable additives protect, preserve, and/or stabilize an RNA (e.g., a capped RNA) during manufacture, storage, and/or administration to a subject. Examples of pharmaceutical acceptable additives include those described in U.S. Patent Publication No. 2017/0119740. Additives may be selected from lipidoids, liposomes, polymers, lipoplexes, peptides, proteins, cells transfected with HCMV RNA vaccines (e.g., for transplantation into a subject), hyaluronidase, nanoparticles (e.g., lipid nanoparticles, core-shell nanoparticles, and/or nanoparticle mimics).

In the context of the present disclosure, a “single-chain RNA capping enzyme” refers to a capping enzyme in which a single polypeptide chain as a monomer displays RNA triphosphatase (TPase), guanylyltransferase (GTase) and guanine-N⁷ methyltransferase (N⁷ MTase) activities. Faustovirus, mimivirus and moumouvirus capping enzymes are examples of single-chain RNA capping enzymes. An example of a single chain RNA capping enzyme is Faustovirus capping enzyme (FCE). For clarity, while vaccinia capping enzyme (VCE) has capping activity, it is a heterodimer and, as such, is not a single-chain RNA capping enzyme.

In the context of the present disclosure, “substitution errors”, with respect to a transcription product made by transcription of a template sequence by an RNA polymerase, refers to positions in the sequence of the transcription product at which the incorporated base does not or cannot form a Watson-Crick pair with the base at the corresponding position in the template sequence. Examples of substitution errors include rA→rC, rA→rU, rA→rG, rC→rA, IC→rU, rC→rG, rU→rA, rU→rC, rU→rG, rG→rA, rG→rG, and rG→rU, where the first letter represents the base that is complementary to the base of the template sequence and the second letter represents the base of the nucleotide that is actually incorporated.

In the context of the present disclosure, “transcription product” refers to a polyribonucleotide product of transcription of polynucleotide having a template by an RNA polymerase. A transcription product may comprise a 5′ untranslated sequence (5′ UTR), a sequence encoding a polypeptide, and/or a 3′ untranslated sequence (3′UTR). A transcription product may be or comprise messenger RNA (mRNA), ribosomal RNA (rRNA), transfer RNAs (tRNAs), small RNA (sRNA), microRNA (miRNA), long non-coding RNA (lncRNA), circular RNA (circRNA), heterogeneous nuclear RNA (hnRNA) or any combination thereof.

In the context of the present disclosure, “uridine analog” refers to modified uridine nucleosides including pseudouridine, N¹-methylpseudouridine, 5-methyluridine, 5-methoxy uridine,_2-thiouridine, 2′-O-methyluridine, 3-methyluridine, 5-hydroxyuridine, 1-methylpseduouridine, 4-thiouridine, 2′-O-methylpseudouridine, 2′-O-methyluridine, and 5-methyl-2-thiouridinc.

All publications, patents, and patent applications mentioned in this specification are herein incorporated by reference to the same extent as if each individual publication, patent, or patent application was specifically and individually indicated to be incorporated by reference. Reagents referenced in this disclosure may be made using available materials and techniques, obtained from the indicated source, and/or obtained from New England Biolabs, Inc. (Ipswich, MA).

The use of synthetic mRNA-based vaccines and therapeutics have been evaluated for over three decades. However, the successful implementation of this modality has been hindered due to the instability of the synthetic mRNA molecule, it's recognition by the cellular immune receptors, as well as the lack of an efficient delivery vehicle. Even though the expression of an antigen from the synthetic mRNA has allowed for effective immune activation as well as antigen presentation, the use of this modality for therapeutics may require the synthetic molecule to be devoid of any immunostimulatory activity. Synthetic mRNAs for therapeutic applications may benefit from methods of high fidelity production and reducing/eliminating by-products that may result in unwanted immune responses. The fidelity with which modifications are incorporated in the mRNAs can affect the efficacy of the drug substance. Enzymatic synthesis processes used to generate mRNA molecules may impact the immune response observed in vivo. Rationalized design of the synthetic mRNAs, in vitro transcription reaction engineering, together with downstream processing of the synthetic mRNA preparations have helped ameliorate some of these effects. Combining chemical modifications of the synthetic mRNA with downstream purification of the mRNA preparation has become the standard for achieving efficient expression from the synthetic molecules while overcoming the immune responses. Among the chemical modifications that are routinely introduced in synthetic mRNAs, w and m1ψ are favored for their ability to suppress an immune response as well as the ability to increase the translation from the mRNAs.

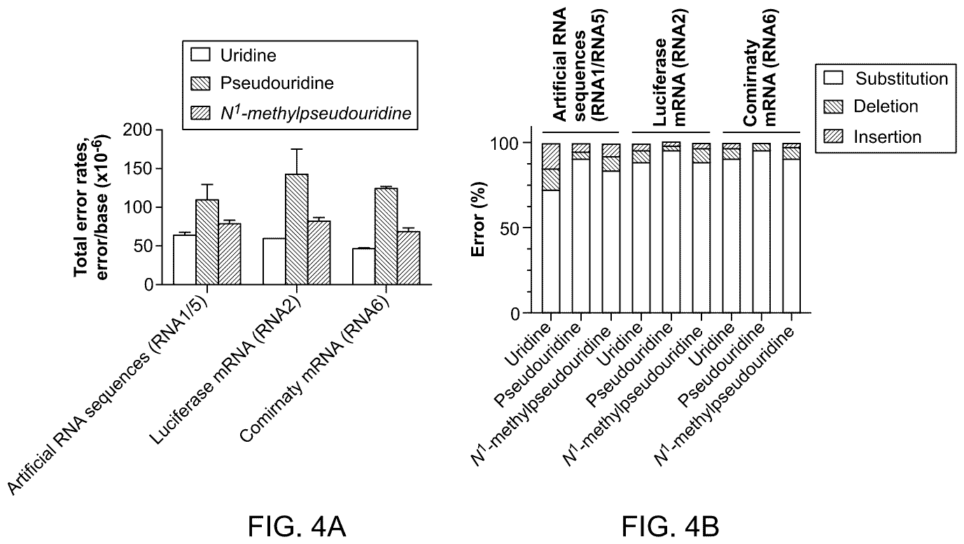

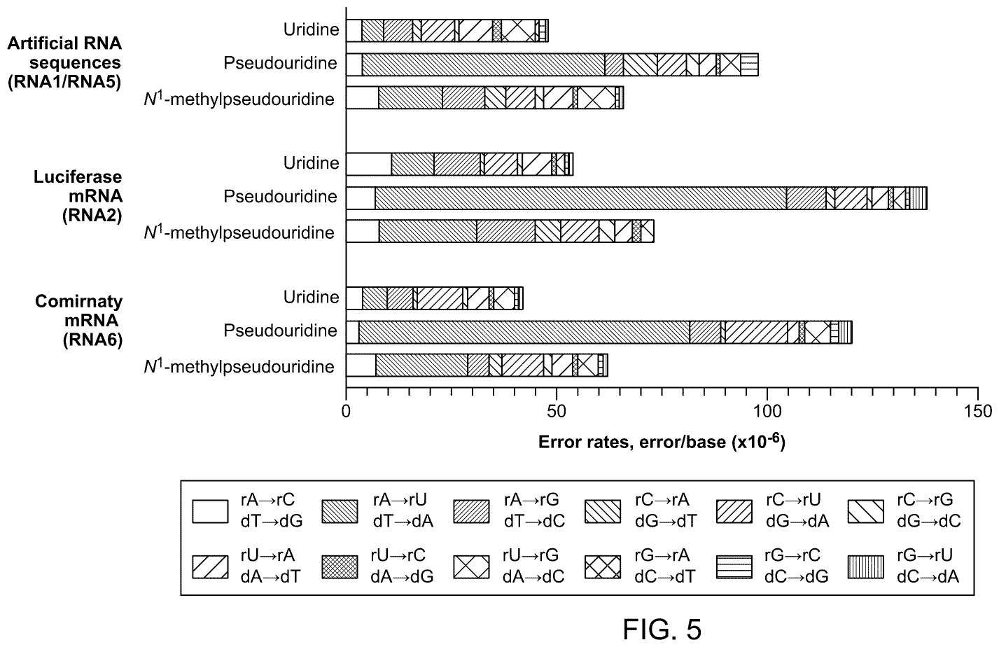

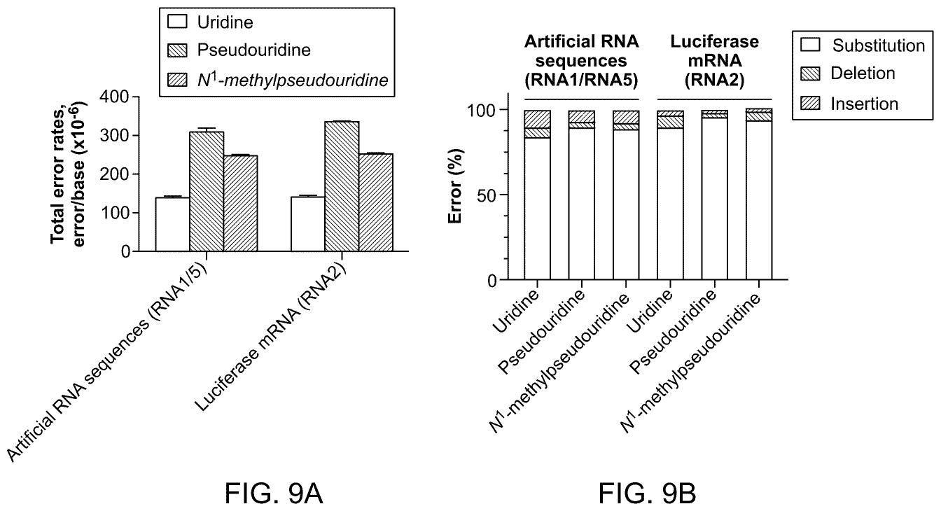

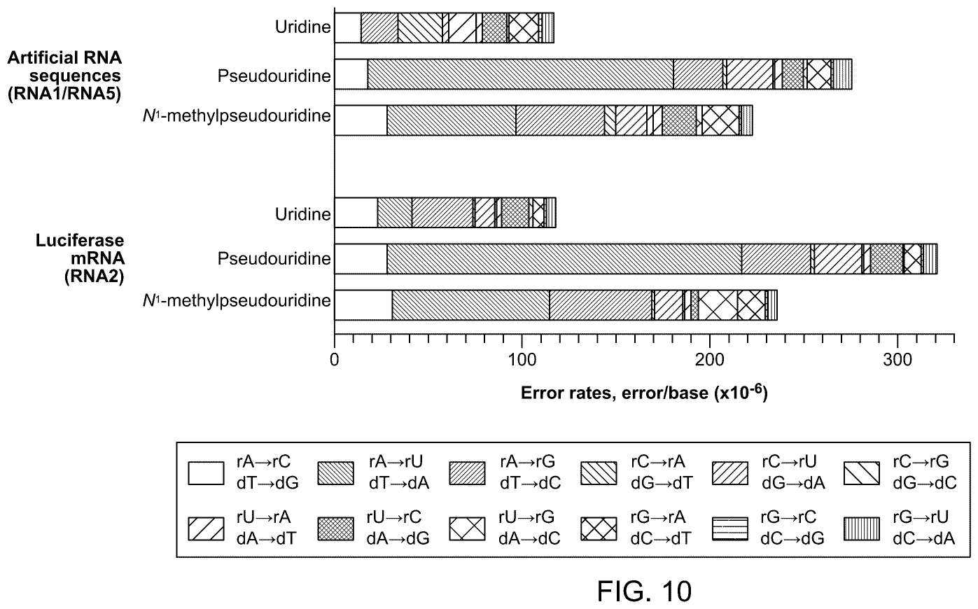

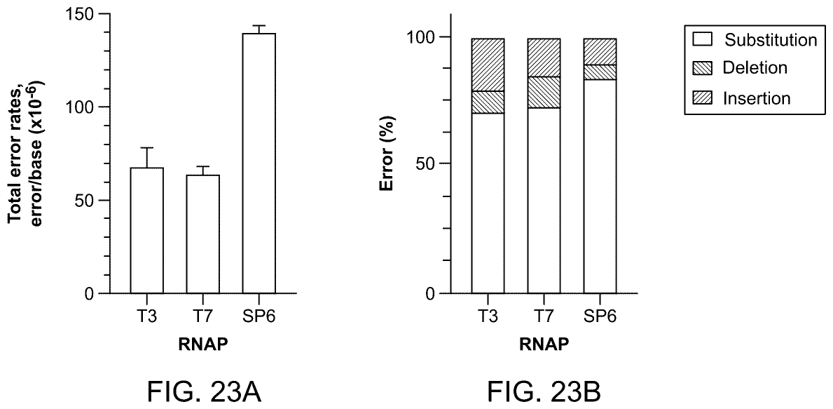

Introducing modifications in synthetic mRNAs may involve replacing a canonical nucleotide with a modified analog during in vitro transcription by ssRNAPs. T7 and SP6 RNAPs are the most commonly used RNA polymerases for in vitro transcription. Both T7 and SP6 have their own promoter specificities and it is also well known that they result in heterogeneous RNA populations. Examples disclosed herein evaluate whether or not these two RNA polymerases incorporate modified nucleotides with similar fidelitics. Data arising from Examples 9 and 10 demonstrate that the combined error rates in RNAs synthesized with SP6 RNAP were up to two-fold higher than those observed with T7 RNAP (FIG. 4, FIG. 5, FIG. 9, and FIG. 10). On the other hand, T3 RNAP which has 82% sequence identity to T7 exhibited error rates comparable to that of T7 (FIG. 23). That said, it is interesting to note that the error profiles of uridine-modified RNA for both T7 and SP6 polymerases were similar. For both RNAPs, the incorporation of y or m1ψ was more error prone than canonical uridinc incorporation (FIG. 4, FIG. 5, FIG. 9, and FIG. 10). However, when uridine analogs were present in the reaction, both T7 and SP6 RNAPs had a very similar substitution error profile with rA→rU substitution being the predominant error observed during in vitro transcription (FIG. 5 and FIG. 10). Available crystal structure capturing T7 RNAP during transcription elongation, as well as biochemical assays and molecular dynamics simulation, have identified several key residues that contribute to transcription fidelity. For example, Tyrosine 639 impacts the pre-insertion of correct nucleotides opposite to the template DNA strand. Since the differences in the total combined errors between T7 and SP6 RNAPs were observed for the same template sequences, and since base pairing between incoming rNTP and DNA template is the same, the fidelity differences of the two enzymes must be associated with differences in the protein sequence, e.g. residues which function to ensure correct base pairing. Interestingly, Tyr639 is conserved in both T7 and SP6 RNAPs, and indeed strictly conserved among bacteriophage ssRNAPs, but neighboring amino acids are not. Residues outside of the active site, which differ between T7 and SP6, may impact fidelity of incorporation and high-fidelity in vitro transcription systems may include modification(s) to one or more such residues. Other high-fidelity in vitro transcription systems may include homologous single-subunit RNA polymerases that have nucleotide incorporation fidelity profiles overlapping with or distinct from that of the RNAPs evaluated in the Examples. Non rA→rU substitution errors may be prevalent in other ssRNAPs and altering the corresponding rNTPs may result in increased fidelity of nucleotide incorporation. Other single-subunit RNAPs may also have sequence determinants conferring increased nucleotide incorporation fidelity that may be incorporated into known RNAPs to increase their nucleotide incorporation fidelity. In addition to targeted amino acid replacements in T7 or SP6 RNAP, sequence determinants of transcriptional fidelity revealed by error rates of homologous ssRNAPs within this protein family may be exploited to further increase fidelity.

Even though the combined error rates observed for the same set of RNA sequences were significantly different between T7 and SP6 RNAPs, there were a few similarities. First, the fidelity with which the analogs were incorporated, followed the same trend-uridine>m1ψ>ψ—with m1ψ-modified RNA exhibiting lower total errors and rA→rU/dT→dA substitution than ψ-modified RNA (FIG. 5 and FIG. 10). Second, the difference in fidelity between uridine-containing RNAs and w/m1ψ-modified RNAs is mainly attributable to a higher rA→rU/dT→dA substitution error (FIG. 5 and FIG. 10). Based on data from multiple templates and reaction conditions, both w and m1ψ have a higher propensity to mis-pair with dT in the template DNA during in vitro transcription. Biophysical studies to measure the melting temperatures (Tm) of synthetic RNA duplexes containing either uridine, w or m1ψ, have shown that both ψ- or m1ψ-containing duplexes have a higher Tm than uridine-containing duplexes. Increased base pairing and stacking has been postulated to contribute to the increased Tm observed. One of the features of w and m1ψ is the C5-C′1 bond that enables rotation between the sugar moiety and the nucleobase, which, in contrast to canonical uridine, could provide improved base pairing and stacking. As compared to uridine and m1ψ, y contains an extra hydrogen bond donor group (NIH) that imparts a universal base character to ψ. Hence, w can not only pair A but can also wobble base-pair with G, U, or C. On the other hand, m1ψ has a methyl group in the N¹-position and therefore does not have the extra hydrogen bond donor and therefore wobble pairing with other nucleotides is not favored. It is plausible that the lower error rates observed in m1ψ-containing RNAs is due to the lack of the extra hydrogen bond donor. Additionally, it is likely that the methyl group in the N¹ position of m1ψ may alter the polarity of the C2 position and make it less favorable to pair with dT in the DNA template. It is also worth noting that stability difference between RNA duplex containing four consecutive w's and m1ψ's was not distinguishable. Basc-pairing differences observed in ExampleS 9 and 10 may be distinguished using stability measurements in which a single rψTP or rm1ψTP pairs with DNA template to mimic the errors observed during in vitro transcription.

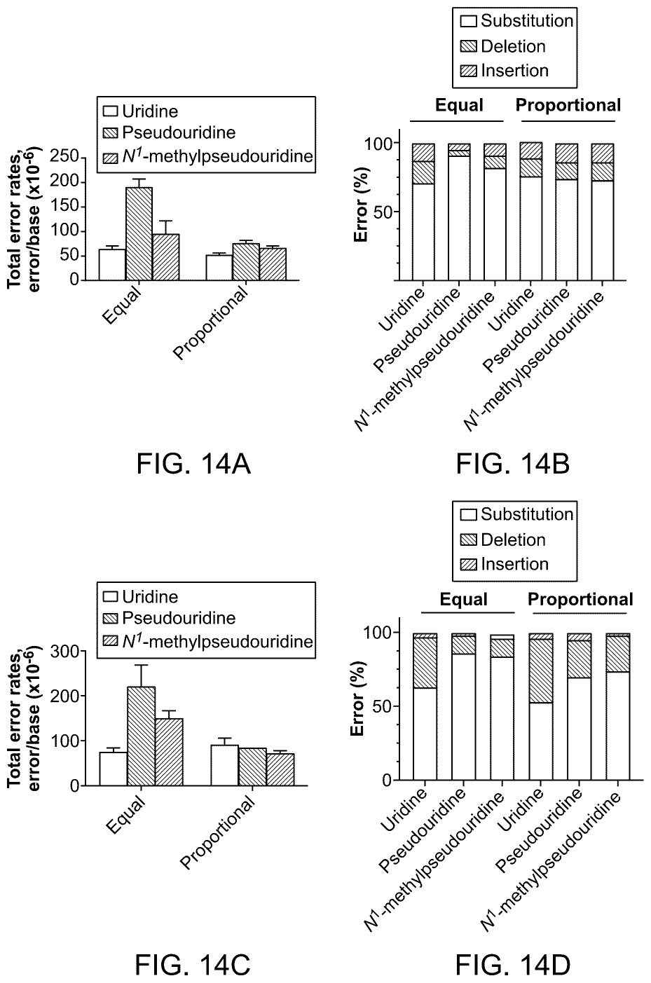

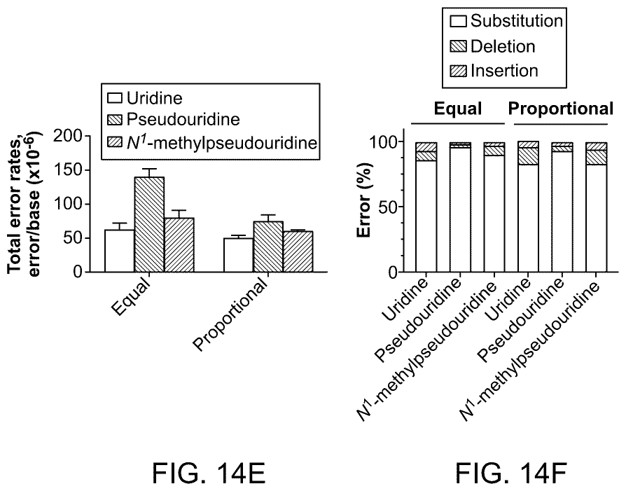

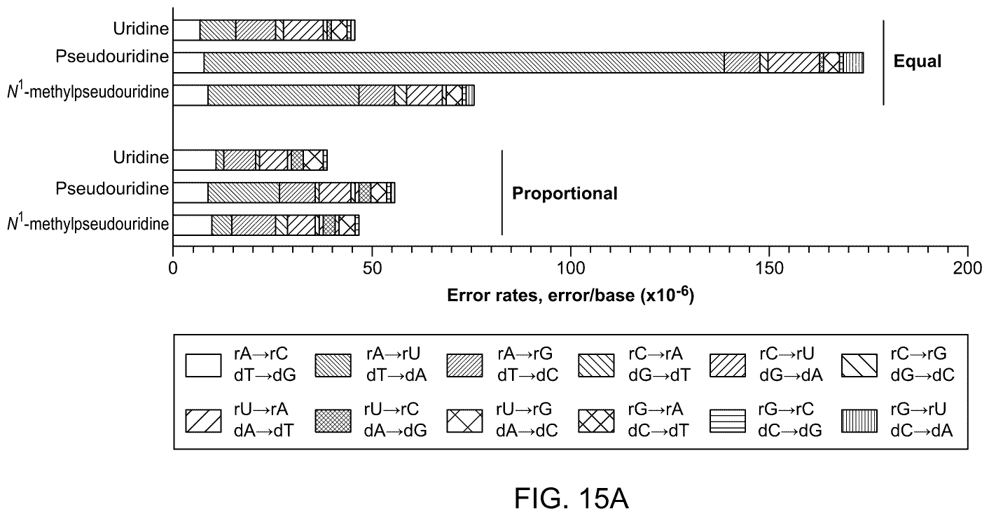

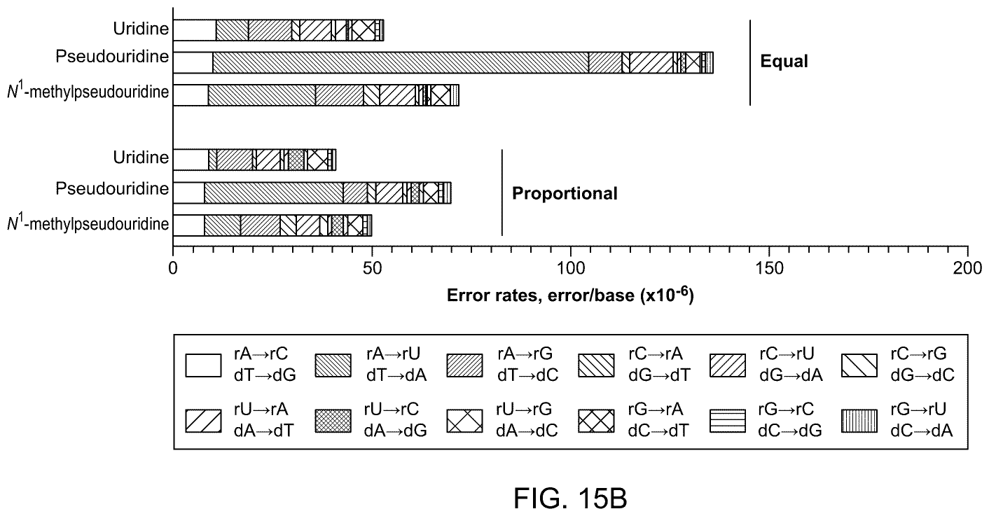

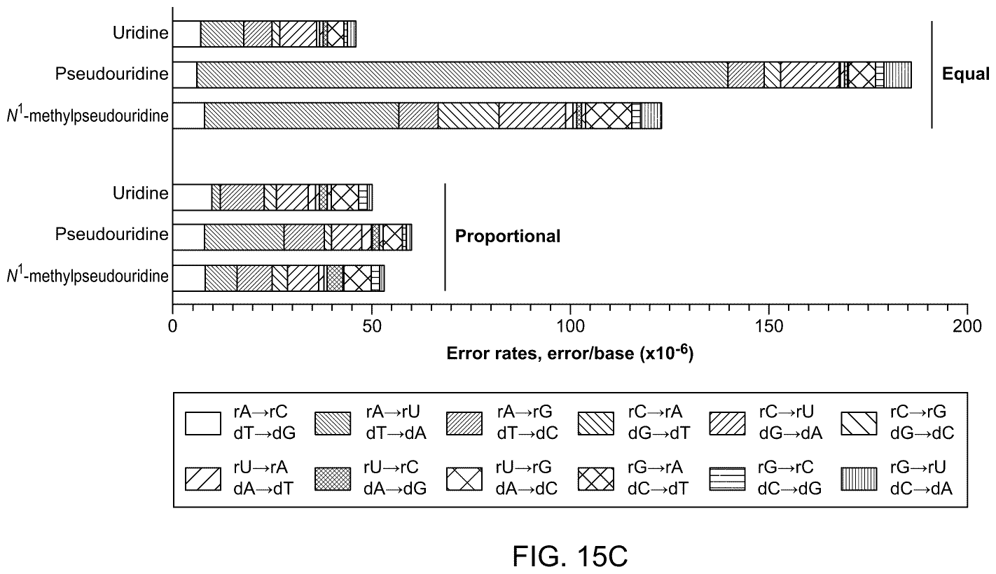

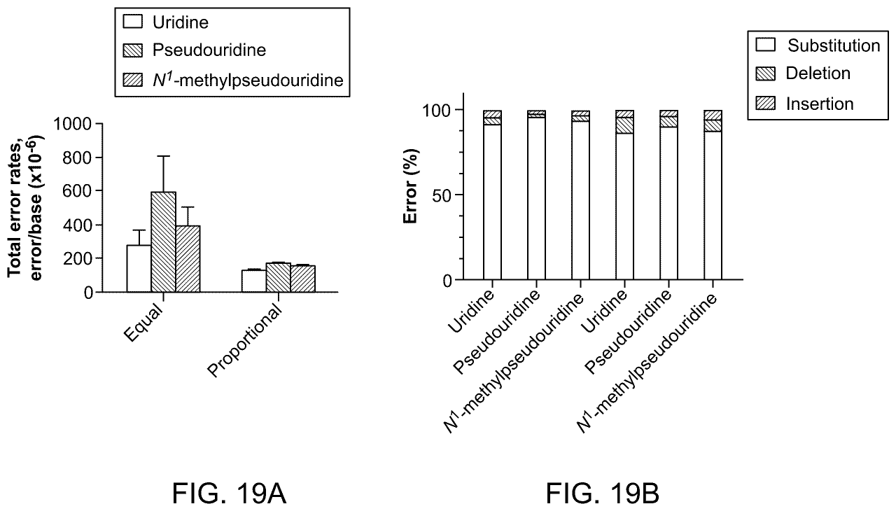

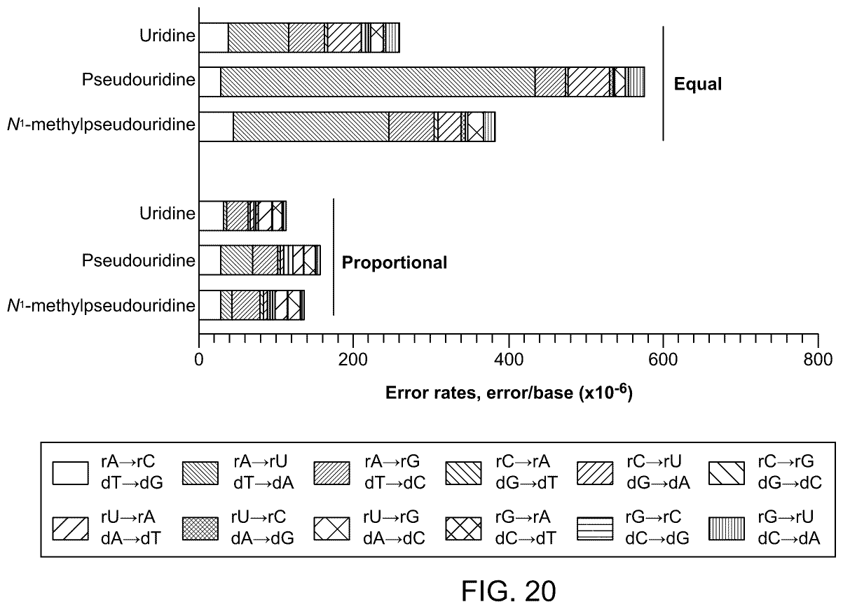

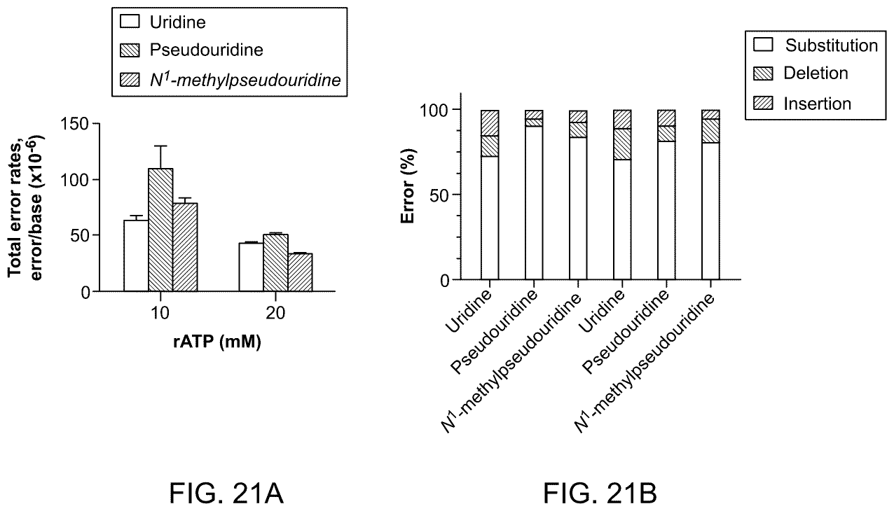

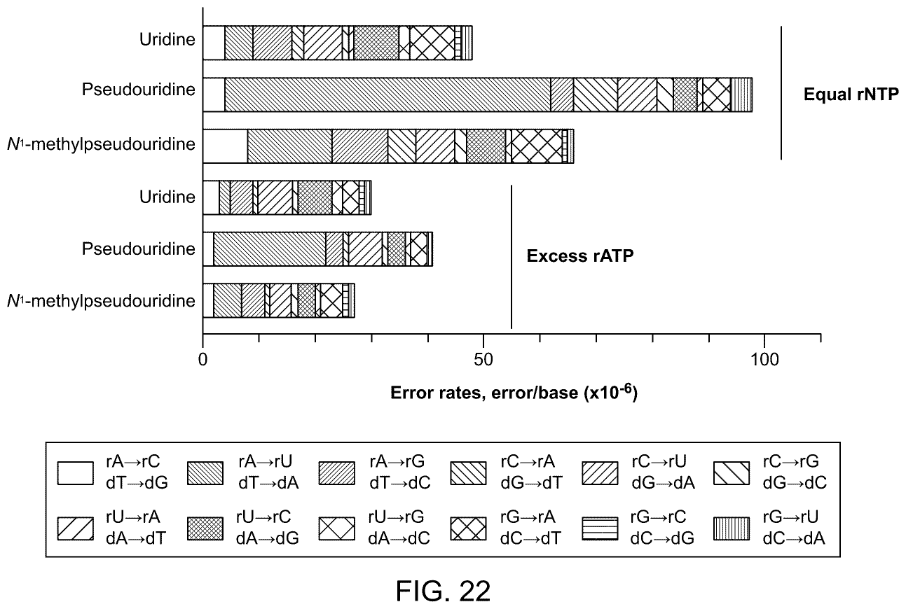

Another aspect of rationalized mRNA design that has been gaining traction has been to reduce the uridine composition without altering the amino acid sequence of the protein encoded from the synthetic mRNA. Uridine depletion in Cas9 mRNA sequence demonstrated a reduction of innate immune response and an increase in Cas9 activity. Comirnaty and Spikevax sequences consist of 19% and 15% uridine, respectively, as compared to the wild-type spike protein sequence that has 33% uridine in the sequence. Often, rationalized design of the synthetic mRNA molecule is further combined with reaction optimization such as altering the rNTP concentrations in the reaction to optimize the RNA yields from the reactions as well as reduction of dsRNA byproducts. Data disclosed here demonstrates that combining uridinc depletion of the RNA sequence with altering the rNTP composition of the reaction, reduces the rA→rU substitutions that are introduced during in vitro transcription (FIG. 14, FIG. 15, FIG. 19, FIG. 20, FIG. 21, and FIG. 22).

The observation that the prevalent rA→rU substitutions introduced during in vitro transcription can be reduced by balancing the rNTPs in the reaction is consistent with a few possible mechanisms for introduction of these substitution errors. By limiting the rψTP amounts or competing with excess rATP, the rA→rU substitution can be altered to reduce the error rates without affecting any of the other substitution errors (FIG. 14, FIG. 15, FIG. 19, FIG. 20, FIG. 21, and FIG. 22). Without being limited to any theory or mechanism of action, mispairing events may occur (e.g., with increased frequency) in the presence of excess rNTPs in the reaction. This is consistent with the overall increased error profile observed in experiments in which the template sequence was intentionally biased to have reduced uridinc (RNA7) and equal molar rNTPs were included in the reaction (10 mM each) relative to templates that had comparable nucleotide usage (RNA2). For example, a one-fold decrease was observed in total combined error in w-incorporated 5.5%-U containing RNA7 compared to 25%-U containing RNA1/RNA5 (FIG. 4 and FIG. 14). Interestingly, the nucleotide composition of RNA1 and RNA5 have equal representation of all the four nucleotides and prevalent rA→rU substitutions were still observed when equal molar rNTPs were added in the reaction. These reactions were performed with 10 mM rNTP each. Presence of excess of rNTPs in the commonly used high-yield transcription reactions could account for this observation. Misincorporations may be more prevalent in the early or late stage of the in vitro transcription process. In some embodiments, an in vitro transcription system may include compositions that limit the initial rNTP concentration in fluid communication with an input line to allow for a steady (e.g., optimized) rNTP feeding mechanism to improve the fidelity of nucleotide incorporation and produce high-fidelity synthetic mRNAs.

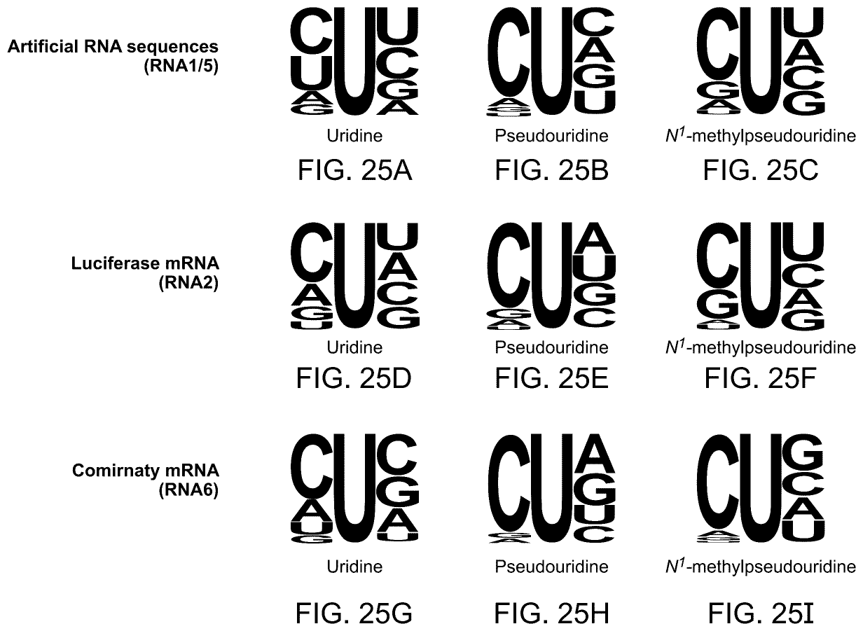

To achieve high-fidelity RNA products, it is desirable to understand the principles of nucleotide incorporation so that, for example, sequences that are more error prone, if any, can be omitted from the synthetic mRNA during design. Comparison of multiple sequence contexts here demonstrated that, other than T7 RNAP-incorporated ψ-modified RNAs that demonstrated slight sequence context preference, there is no strong correlation between the sequence context of the DNA template and the substitution errors observed under any condition tested (FIG. 25). Furthermore, the errors observed were distributed throughout of the length of RNA did not affect the misincorporation events that were observed when in vitro transcription was performed with either T7 RNAP or SP6 RNAP and when uridine analogs were used.

Results disclosed here demonstrate that the presence of y and m1ψ in the in vitro transcription reactions result in higher base substitution errors in the modified RNAs. Errors might affect the efficacy, tolerance, and/or safety of the synthetic mRNA drug substance in vivo. For example, errors (e.g., mismatch errors) in in vitro transcribed mRNAs comprising modified nucleotides throughout the body of the mRNA may impact translation fidelity in cells. In human embryonic kidney cell, low frequency translation elongation miscoding events are observed from w-containing mRNAs due to altered tRNA selection in w-containing codons. For m1ψ-substituted RNAs, it has been shown that translation initiation and ribosome transit is altered in vivo. Understanding what errors are incorporated during the RNA synthesis process and how that further affects the identity of the protein synthesized may help therapeutic applications, for example, applications that may require repeat dosing of the mRNAs and/or expression of the protein of choice. To predict the performance of these synthetic molecules, it is desirable to understand where variability comes from and to be able to define the rules to avoid these variabilities.

According to some embodiments, methods of improving fidelity of RNA synthesis may comprise contacting an RNA polymerase, a polynucleotide (e.g., a polynucleotide comprising a template sequence), and a composition comprising (e.g., two or more) rNTPs in a ratio other than an equimolar ratio to form a transcription product, wherein the transcription product comprises fewer base misincorporation errors than a transcription product arising from contacting the RNA polymerase, the polynucleotide, and a composition comprising the same rNTPs in an equimolar ratio. The ratio of rNTPs may be selected in light of the RNA polymerase, the errors to be avoided, and/or the composition of the RNA template.

According to some embodiments, selection of an RNA polymerase may impact the fidelity of nucleotide incorporation. For example, an RNA polymerase with an observed pattern of misincorporation with equimolar rNTPs may be selected in light of a template sequence to be used where the ratio of bases in a transcription product or other sequence information indicates that there will be limited opportunities for the observed misincorporation. Remaining misincorporation may be mitigated, in some embodiments, by modifying the ratio of rNTPs used in transcription reactions in accordance with the present disclosure.

The ratio of nucleotides in a sequence complementary to an RNA template may be expressed as follows:
wJ: xK: yL: zM,
   wherein
ψ, x, y, and z are each independently positive numbers from 0-50,
J is adenosine or an adenosine analog,
K is uridine or a uridine analog,
L is guanosine or a guanosine analog, and
M is cytidine or a cytidine analog.
   Similarly, the ratio of rNTPs in a composition comprising rNTPs (e.g., an RNA synthesis reaction or a composition otherwise for use in connection with RNA synthesis) may be expressed as follows:
w′JTP: x′KTP: y′LTP: z′MTP,
   wherein
w′, x′, y′, and z′ are each independently positive numbers from 0-50 (where 0 indicates the referenced rNTP is not present),
optionally, up to three of w′, x′, y′, and z′ may be equal to one another±10% (e.g., may be equal to one another),
J is adenosine or an adenosine analog,
K is uridine or a uridine analog,
L is guanosine or a guanosine analog, and
M is cytidine or a cytidine analog.
   For clarity, in this context, zero is regarded as a positive number. In some embodiments the ratio of bases in a sequence complementary to a template (e.g., ψ, x, y, and z are each independently numbers from 0-50) and/or the ratio of rNTPs in a transcription reaction (e.g., w′, x′, y′, and z′ are each independently positive numbers from 0-50) may be selected in contemplation of a transcription product (e.g., an mRNA or protein) having a therapeutic or other physiological effect. In some embodiments, other ratios may be desirable. For example, ψ, x, y, and z each independently may be numbers from 0-100 and/or w′, x′, y′, and z′ each independently may be numbers from 0-100.

In some embodiments, at least 2 of, at least 3 of, or all 4 of w′, x′, y′, and z′ are greater than zero. Methods of synthesizing RNA, according to some embodiments, may comprise contacting an RNA template, an RNA polymerase, and a composition comprising ribonucleotide triphosphates, wherein at least one of w′, x′, y′, and z′ is not equal to at least one of the others of w′, x′, y′, and z′. For example, w′ may be at least 1.01× more than x′, y′, and/or z′; w′ may be at least 1.02× more than x′, y′, and/or z′; w′ may be at least 1.03× more than x′, y′, and/or z′; w′ may be at least 1.06× more than x′, y′, and/or z′; w′ may be at least 1.1× more than x′, y′, and/or z′; w′ may be at least 1.15× more than x′, y′, and/or z′; w′ may be at least 1.2× more than x′, y′, and/or z′; w′ may be at least 1.25× more than x′, y′, and/or z′; w′ may be at least 1.3× more than x′, y′, and/or z′; w′ may be at least 1.35× more than x′, y′, and/or z′; w′ may be at least 1.4× more than x′, y′, and/or z′; w′ may be at least 1.45× more than x′, y′, and/or z′; w′ may be at least 1.5× more than x′, y′, and/or z′; w′ may be at least 1.55× more than x′, y′, and/or z′; w′ may be at least 1.6× more than x′, y′, and/or z′; w′ may be at least 1.65× more than x′, y′, and/or z′; w′ may be at least 1.7× more than x′, y′, and/or z′; w′ may be at least 1.75× more than x′, y′, and/or z′; w′ may be at least 1.8× more than x′, y′, and/or z′; w′ may be at least 1.85× more than x′, y′, and/or z′; w′ may be at least 1.9× more than x′, y′, and/or z′; w′ may be at least 1.95× more than x′, y′, and/or z′; w′ may be at least 2× more than x′, y′, and/or z′; w′ may be at least 2.5× more than x′, y′, and/or z′; w′ may be at least 3× more than x′, y′, and/or z′; w′ may be at least 3.5× more than x′, y′, and/or z′; w′ may be at least 4× more than x′, y′, and/or z′; w′ may be at least 4.5× more than x′, y′, and/or z′; w′ may be at least 5× more than x′, y′, and/or z′; w′ may be at least 10× more than x′, y′, and/or z′; w′ may be at least 25× more than x′, y′, and/or z′; and/or w′ may be at least 50× more than x′, y′, and/or z′.

For example, x′ may be at least 1.01× more than w′, y′, and/or z′; x′ may be at least 1.02× more than w′, y′, and/or z′; x′ may be at least 1.03× more than w′, y′, and/or z′; x′ may be at least 1.06× more than w′, y′, and/or z′; x′ may be at least 1.1× more than w′, y′, and/or z′; x′ may be at least 1.15× more than w′, y′, and/or z′; x′ may be at least 1.2× more than w′, y′, and/or z′; x′ may be at least 1.25× more than w′, y′, and/or z′; x′ may be at least 1.3× more than w′, y′, and/or z′; x′ may be at least 1.35× more than w′, y′, and/or z′; x′ may be at least 1.4× more than w′, y′, and/or z′; x′ may be at least 1.45× more than w′, y′, and/or z′; x′ may be at least 1.5× more than w′, y′, and/or z′; x′ may be at least 1.55× more than w′, y′, and/or z′; x′ may be at least 1.6× more than w′, y′, and/or z′; x′ may be at least 1.65× more than w′, y′, and/or z′; x′ may be at least 1.7× more than w′, y′, and/or z′; x′ may be at least 1.75× more than w′, y′, and/or z′; x′ may be at least 1.8× more than w′, y′, and/or z′; x′ may be at least 1.85× more than w′, y′, and/or z′; x′ may be at least 1.9× more than w′, y′, and/or z′; x′ may be at least 1.95× more than w′, y′, and/or z′; x′ may be at least 2× more than w′, y′, and/or z′; x′ may be at least 2.5× more than w′, y′, and/or z′; x′ may be at least 3× more than w′, y′, and/or z′; x′ may be at least 3.5× more than w′, y′, and/or z′; x′ may be at least 4× more than w′, y′, and/or z′; x′ may be at least 4.5× more than w′, y′, and/or z′; x′ may be at least 5× more than w′, y′, and/or z′; x′ may be at least 10× more than w′, y′, and/or z′; x′ may be at least 25× more than w′, y′, and/or z′; and/or x′ may be at least 50× more than w′, y′, and/or z′.

For example, y′ may be at least 1.01× more than w′, x′, and/or z′; y′ may be at least 1.02× more than w′, x′, and/or z′; y′ may be at least 1.03× more than w′, x′, and/or z′; y′ may be at least 1.06× more than w′, x′, and/or z′; y′ may be at least 1.1× more than w′, x′, and/or z′; y′ may be at least 1.15× more than w′, x′, and/or z′; y′ may be at least 1.2× more than w′, x′, and/or z′; y′ may be at least 1.25× more than w′, x′, and/or z′; y′ may be at least 1.3× more than w′, x′, and/or z′; y′ may be at least 1.35× more than w′, x′, and/or z′; y′ may be at least 1.4× more than w′, x′, and/or z′; y′ may be at least 1.45× more than w′, x′, and/or z′; y′ may be at least 1.5× more than w′, x′, and/or z′; y′ may be at least 1.55× more than w′, x′, and/or z′; y′ may be at least 1.6× more than w′, x′, and/or z′; y′ may be at least 1.65× more than w′, x′, and/or z′; y′ may be at least 1.7× more than w′, x′, and/or z′; y′ may be at least 1.75× more than w′, x′, and/or z′; y′ may be at least 1.8× more than w′, x′, and/or z′; y′ may be at least 1.85× more than w′, x′, and/or z′; y′ may be at least 1.9× more than w′, x′, and/or z′; y′ may be at least 1.95× more than w′, x′, and/or z′; y′ may be at least 2× more than w′, x′, and/or z′; y′ may be at least 2.5× more than w′, x′, and/or z′; y′ may be at least 3× more than w′, x′, and/or z′; y′ may be at least 3.5× more than w′, x′, and/or z′; y′ may be at least 4× more than w′, x′, and/or z′; y′ may be at least 4.5× more than w′, x′, and/or z′; y′ may be at least 5× more than w′, x′, and/or z′; y′ may be at least 10× more than w′, x′, and/or z′; y′ may be at least 25× more than w′, x′, and/or z′; and/or y′ may be at least 50× more than w′, x′, and/or z′.

For example, z′ may be at least 1.01× more than w′, x′, and/or y′; z′ may be at least 1.02× more than w′, x′, and/or y′; z′ may be at least 1.03× more than w′, x′, and/or y′; z′ may be at least 1.06× more than w′, x′, and/or y′; z′ may be at least 1.1× more than w′, x′, and/or y′; z′ may be at least 1.15× more than w′, x′, and/or y′; z′ may be at least 1.2× more than w′, x′, and/or y′; z′ may be at least 1.25× more than w′, x′, and/or y′; z′ may be at least 1.3× more than w′, x′, and/or y′; z′ may be at least 1.35× more than w′, x′, and/or y′; z′ may be at least 1.4× more than w′, x′, and/or y′; z′ may be at least 1.45× more than w′, x′, and/or y′; z′ may be at least 1.5× more than w′, x′, and/or y′; z′ may be at least 1.55× more than w′, x′, and/or y′; z′ may be at least 1.6× more than w′, x′, and/or y′; z′ may be at least 1.65× more than w′, x′, and/or y′; z′ may be at least 1.7× more than w′, x′, and/or y′; z′ may be at least 1.75× more than w′, x′, and/or y′; z′ may be at least 1.8× more than w′, x′, and/or y′; z′ may be at least 1.85× more than w′, x′, and/or y′; z′ may be at least 1.9× more than w′, x′, and/or y′; z′ may be at least 1.95× more than w′, x′, and/or y′; z′ may be at least 2× more than w′, x′, and/or y′; z′ may be at least 2.5× more than w′, x′, and/or y′; z′ may be at least 3× more than w′, x′, and/or y′; z′ may be at least 3.5× more than w′, x′, and/or y′; z′ may be at least 4× more than w′, x′, and/or y′; z′ may be at least 4.5× more than w′, x′, and/or y′; z′ may be at least 5× more than w′, x′, and/or y′; z′ may be at least 10× more than w′, x′, and/or y′; z′ may be at least 25× more than w′, x′, and/or y′; and/or z′ may be at least 50× more than w′, x′, and/or y′.

According to some embodiments, the ratio of one rNTP to the other rNTPs present may be increased or decreased in light of observed misincorporation frequencies. For example, a ratio of a nucleotide to the others may be increased where it is observed that the nucleotide is not incorporated into a transcription product as often as it should according to the template used. A ratio of a nucleotide to the others may be decreased where it is observed that the nucleotide is incorporated into a transcription product more often than it should according to the template used. For example, if rA→rU substitutions are observed with a given polymerase and template using a 1:1:1:1 ratio of rNTPs, a method of making an RNA may comprise contacting the RNA template, the polymerase, and a composition comprising ATP, UTP, GTP and CTP at a ratio of w′: x′: y′: z′, wherein x′ is 1 and w′, y′, and z′ are each, independently, 1.01-5 (e.g., 2-4).

In some embodiments, the ratio of rNTPs used for RNA synthesis may be selected in light of the base composition of the predicted sequence of a transcription product of the selected template. For example, the ratio of rNTPs used for RNA synthesis may be referred to as “proportional” to the ratio of bases in the sequence complementary to (e.g., encoded by) a template sequence where the ratio of rNTPs corresponds to the base composition of the predicted transcription product of a template sequence. For example,
w may equal w′±10%,
x may equal x′±10%,
y may equal y′±10%, and/or
z may equal z′±10%,
   provided that w′, x′, y′, and z′ are not equal to each other. For example, ψ, x, y, and z may equal w′, x′, y′, and z′, respectively, provided that w′, x′, y′, and z′ are not equal to each other. For example,
w may equal w′±1%, w may equal w′±2%, w may equal w′±3%, w may equal w′±4%, w may equal w′±5%, w may equal w′±7%, w may equal w′±10%, and/or w may equal w′±20%;
x may equal x′±1%, x may equal x′±2%, x may equal x′±3%, x may equal x′±4%, x may equal x′±5%, x may equal x′±7%, x may equal x′±10%, and/or x may equal x′±20%;
y may equal y′±1%, y may equal y′±2%, y may equal y′±3%, y may equal y′±4%, y may equal y′±5%, y may equal y′±7%, y may equal y′±10%, and/or y may equal y′±20%; and/or
z may equal z′±1%, z may equal z′±2%, z may equal z′±3%, z may equal z′±4%, z may equal z′±5%, z may equal z′±7%, z may equal z′±10%, and/or z may equal z′±20%,
   provided that w′, x′, y′, and z′ are not equal to each other. To illustrate, a composition may comprise rNTPs having a molar ratio of 27-33 rATP: 4-6 rUTP: 27-33 rGTP: 27-33 rCTP and, in some embodiments, may be contacted with an RNA polymerase and a polynucleotide having a template sequence wherein the molar ratio of bases in an RNA transcribed from the template sequence is 30 A, 5 U (or T if the polynucleotide is DNA), 30 G and 30 C. In this illustration, the rNTPs correspond to or are proportioned to the bases in the encoded sequence. Specifically, w=w′±10%, x=x′±20%, y=y′±10%, and z=z′±10%.

Methods and compositions of the present disclosure, according to some embodiments, allow production of transcription products that may have fewer substitution errors. Transcription products with fewer errors and compositions comprising such transcription products may be used for or included in compositions for research, diagnostic and/or therapeutic purposes. With fewer substitution errors, such transcription products and compositions may better fulfill its intended purpose. For example, transcription products (e.g., IVT products) with greater uniformity/less sequence diversity and compositions comprising such transcription products may be less immunogenic, have more uniform pharmacokinetics and/or pharmacodynamics, and/or have a better safety profile (e.g., when delivered to a human or non-human mammal). Transcription products (e.g., encoding a protein) with fewer errors and compositions comprising such transcription products may be used to prepare proteins having fewer errors. For example, a method may include translating a transcription product with fewer substitution errors to form a polypeptide having an amino acid sequence that better reflects the sequence encoded in the template. According to some embodiments, the present disclosure provides methods and compositions for generating transcription products (e.g., IVT products) comprising fewer or no contaminating transcription products comprising substitution errors.

### EXAMPLES

Some specific example embodiments may be illustrated by one or more of the examples provided herein.

### Example 1: Generation of DNA Templates for In Vitro Transcription (IVT) of Long RNAs

All of the oligonucleotides for in vitro transcription and reverse transcription were synthesized by Integrated DNA Technologies (IDT, Coralville IA). For in vitro transcription reactions with different RNA polymerases, the corresponding promoter sequences were inserted in the DNA templates using Q5 Site-Directed Mutagenesis Kit (E0554, New England Biolabs). DNA templates encoding for functional mRNAs, RNA2 (Cypridina luciferase mRNA; 1707 nucleotides) and RNA3 (part of BNT162b/Comirnaty mRNA; 4187 nucleotides, were synthesized by GenScript Inc. (GenScript, Piscataway NJ) and introduced into standard high-copy plasmids. The plasmids were propagated in E. coli (C2987, New England Biolabs) and purified with Monarch Plasmid Miniprep Kit (T1010, New England Biolabs). Plasmids were digested with restriction enzymes to generate linearized templates for in vitro transcription. The linearized plasmids were treated with PreCR Repair Mix (M0309, New England Biolabs) and purified with Monarch PCR & DNA Cleanup Kit (T1030, New England Biolabs).

### Example 2: In Vitro Transcription (IVT)

In vitro transcription reactions were performed with the high-yield in vitro transcription kits (E2040 and E2070, New England Biolabs, Ipswich, MA), consisting of 40 mM rNTP (pH buffered with sodium phosphate) for T7 RNA polymerase and 20 mM rNTP (pH buffered with Tris) for SP6 RNA polymerase. For modified RNAs, UTP was replaced with either pseudouridine-5′-triphosphate (N-1019, TriLink Biotechnologies, San Diego, CA) or N¹-Methylpseudouridine-5′-Triphosphate (N-1081, TriLink Biotechnologies, San Diego, CA). Linearized plasmid DNA was used as DNA template for in vitro transcription. Linearization was performed with either Hpal, NotI, or Xhol (New England Biolabs, Ipswich, MA). The plasmids used for in vitro transcription also contained the promoter sequences for either T7 RNA polymerase or SP6 RNA polymerase. In vitro transcription reactions were incubated at 37° C. for two hours. Following in vitro transcription, the DNA template was removed with Turbo DNase (AM2238, Thermo Fisher Scientific, Waltham, MA) digestion at 37° C. for 30 minutes and then purified with Monarch RNA Cleanup Kit (T2050, New England Biolabs, Ipswich, MA) for long RNA (1020 nucleotides to 4187 nucleotides).

### Example 3: Bioanalyzer for RNA Size Distribution and Integrity

Eluted RNA samples from the in vitro transcription reactions were diluted based on concentrations measured on a Nanodrop spectrophotometer (13-400-519, Thermo Fisher Scientific) and denatured at 70° C. for 2 minutes and snap-cooled on ice. 250 ng RNA samples were prepared with RNA 6000 Nano kits (5067, Agilent Technologies) and the integrity and the size distribution of the RNA was assessed using mRNA Nano series 2 assay (G2938, Agilent Technologies).

### Example 4: Nucleoside Digestion of RNA and UHPLC-MS Analyses to Assess Modification Incorporation

Purified modified RNA and RNA without any chemical modification were digested with nucleoside digestion mix (M0649, New England Biolabs) at 37° C. for 1 hour. Base composition analysis was performed by Liquid Chromatography-Mass Spectrometry (LC-MS) using an Agilent 1290 Infinity II UHPLC equipped with G7117A Diode Array Detector and 6135XT MS Detector, on a Waters Xselect HSS T3 XP column (2.1×100 mm, 2.5 μm) with the gradient mobile phase consisting of methanol and 10 mM ammonium acetate buffer (pH 4.5).

### Example 5: First and Second Strand cDNA Synthesis for PacBio

The cDNA synthesis was performed as described with a modified cleanup step using Monarch PCR & DNA Cleanup Kit (T1030, New England Biolabs) Potapov et al., Nucleic Acids Res, 2018. 46 (11): p. 5753-5763.

### Example 6: Pacific Biosciences SMRTbell Library Preparation and Sequencing

The library preparation for sequencing on RSII system was performed as described Potapov et al., Nucleic Acids Res, 2018. 46 (11): p. 5753-5763. For the sequencing on the Sequel platform, about 1.5 μg cDNA was treated with NEBNext End Repair Module (E6050, New England Biolabs) at room temperature for 5 minutes, followed by purification with Monarch PCR & DNA Cleanup Kit (T1030, New England Biolabs). The end-repaired cDNA was ligated with 2 μL barcoded adaptor (100-466-000, Pacific Biosciences) with T4 DNA Ligase (M0202, New England Biolabs) in 50 μL reaction volume at room temperature for 1 hour, followed by purification with Monarch PCR & DNA Cleanup Kit (T1030, New England Biolabs). The un-ligated adaptor and cDNA were digested with E. coli Exonuclease III (M0206, New England Biolabs) and Exonuclease VII (M0379, New England Biolabs) in 1X standard Taq buffer at 37° C. for 1 hour, followed by cleaning up with Monarch PCR & DNA Cleanup Kit (T1030, New England Biolabs). The ligated DNA was repaired with PreCR Repair Mix (M0309, New England Biolabs) at 37° C. for 30 minutes. The libraries were purified with 0.6X volume of AMPure PB beads (100-265-900, Pacific Biosciences) and pooled for sequencing runs. SMRT Link was used to generate the protocol for primer annealing, polymerase binding (Sequel Binding Kit 3.0 (101-613-900, Pacific Biosciences)), cleanup and final loading to three SMRT Cells LR and sequencing using Sequel system.

### Example 7: Data Analysis

Analysis of sequencing data was performed as described Potapov et al., Nucleic Acids Res, 2018. 46 (11): p. 5753-5763. In short, high-accuracy consensus sequences were built for the first and second strand for each sequenced double-stranded DNA. The consensus sequences were aligned to the reference sequence and base substitutions, deletions, and insertions were determined. The first strand error rates were determined by comparing the first strand consensus sequences to the reference sequence (RNA strand), and mutations were required to be present in both strands. The average error and standard deviation were then calculated for each template and enzyme: for RNA1 and RNA5 (1122- and 1124-nucleotide synthetic sequences that includes all possible four-base combinations), one measurement was performed and the error rates were combined and referred as RNA1/RNA5. For other RNA templates, two independent repeats were performed. The relative fold change was calculated for each substitution as (M-U)/U, where M is the substitution rate on modified RNA (m1ψ or w) and U is the substitution rate on unmodified RNA.

### Example 8: T7 and SP6 RNAPs Incorporate m1ψEfficiently

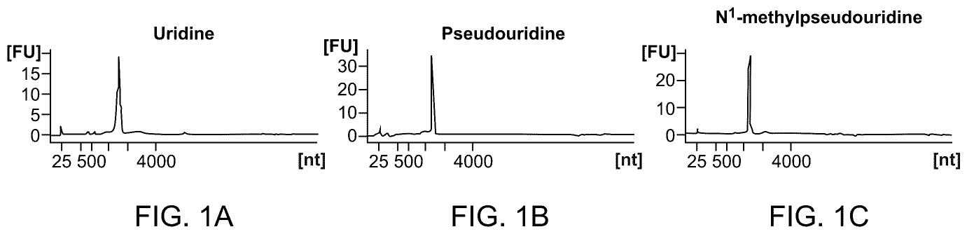

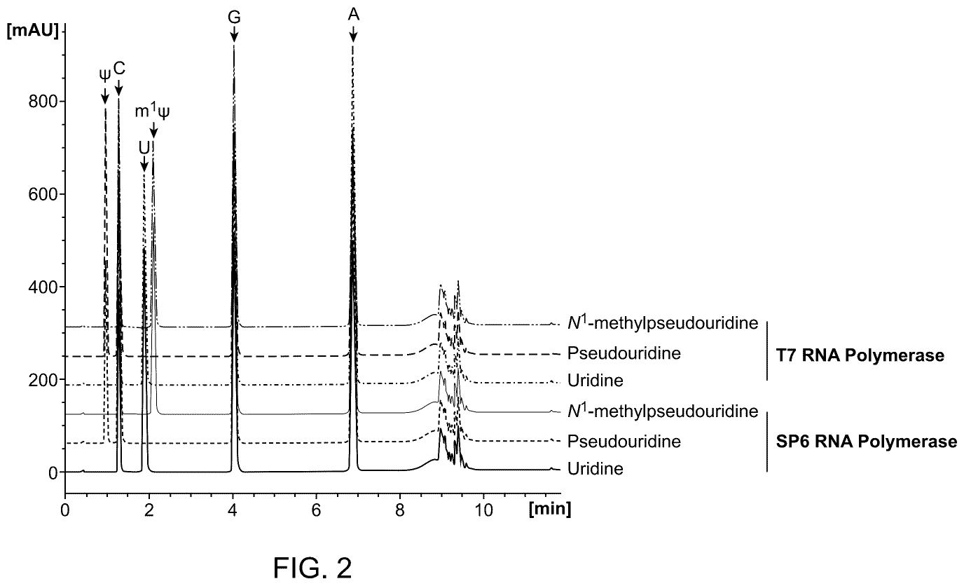

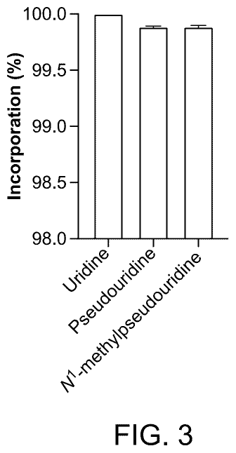

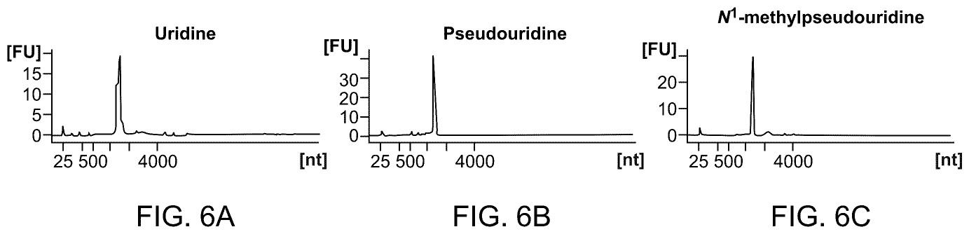

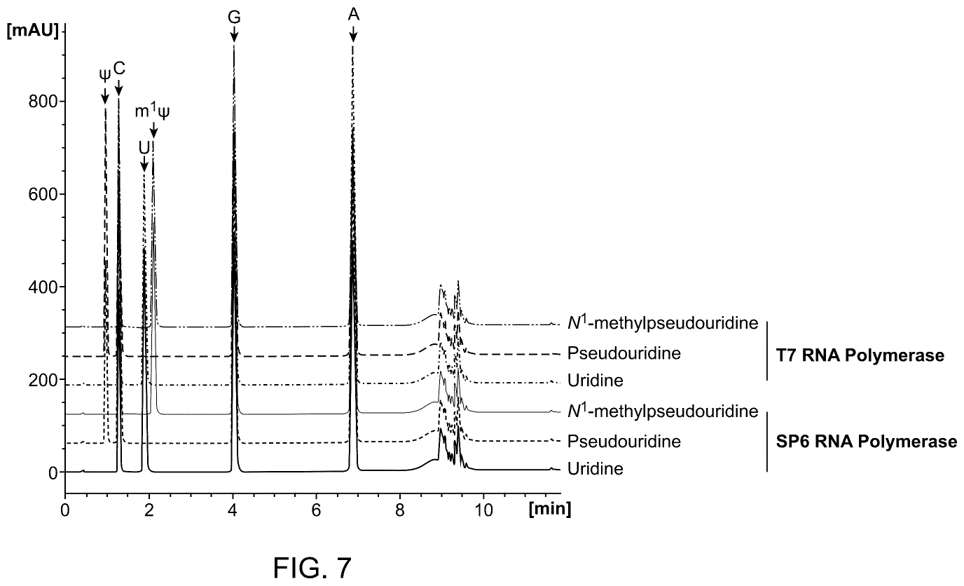

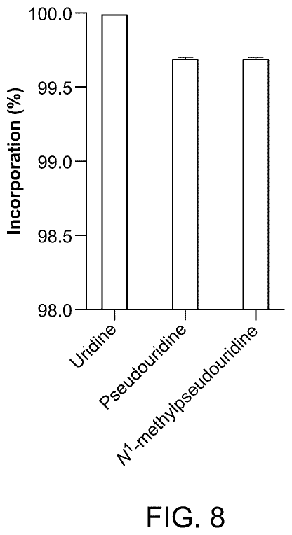

The efficiency of m1ψincorporation during in vitro transcription was investigated in different RNA substrates of varying length and sequence. The base composition of the synthesized RNA was analyzed with ultra-high performance liquid chromatography coupled with mass spectrometry. For the long synthetic RNAs with length ranging from 1122 to 4178 nucleotides, the integrity of the RNA was determined using Bioanalyzer. Synthesis of full-length RNAs of expected sizes were observed in reactions performed in the presence of m1ψ with both T7 RNAP and SP6 RNAP (FIG. 1 and FIG. 6). Furthermore, similar to ψ, total m1ψTP incorporation was as expected in RNA2 (a Cypridina luciferase mRNA) when in vitro transcription was performed with T7 or SP6 RNAP (FIG. 2, FIG. 3, FIG. 7, and FIG. 8). In that light, similar to w and uridine, m1ψ may be incorporated efficiently during in vitro transcription and result in synthesis of full-length run-off products.

### Example 9: m1ψ is Incorporated with Higher Fidelity than w by T7 RNAP

To determine the fidelity of m1ψincorporation during in vitro transcription, the Pacific Biosciences Single Molecule Real-Time (SMRT) sequencing-based assay described by Potapov et al 2018 was adapted to the PacBio Sequel I system to enable higher sequencing capacity and multiplexing. The in vitro transcribed RNAs were reverse transcribed into double-stranded cDNA using ProtoScript II reverse transcriptase (RT) and sequenced in the SMRT sequencing platform. Errors in the first strand that stem from combined RNAP and RT error, referred hereafter as combined errors, were analyzed. The combined errors in two synthetic sequences (RNA1 and RNA5) that represent all possible four-base combinations (templates described as DNA-1 and DNA-2 (Potapov et al., Nucleic Acids Res, 2018. 46 (11): p. 5753-5763) were determined first. The error rates of pooled RNA1 and RNA5 (referred as RNA1/RNA5) in reactions with canonical uridine, using the Sequel I system was observed to be 6.4±0.4×10⁵ error/base (FIG. 4) as compared to 5.6±0.8×10⁻⁵ error/base using the PacBio RSII system. Since the combined error rates were comparable between the two platforms, the Sequel I system was used for the subsequent experiments.

In vitro transcription error rates were tested next for reactions with m1ψ for RNA1 and RNA5 sequences performed with T7 RNAP under standard high-yield reaction conditions. Total combined error rates in m1ψ-containing reactions were observed to be 8.0±0.3×10⁻⁵ as compared to 6.4±0.4×10⁻⁵ error/base for uridine-containing reactions and 1.1±0.2×10⁻⁴ errors/base for w-containing reactions (FIG. 4). The combined error rates in ψ-modified RNAs were observed to be greater than unmodified RNAs, while that in m1ψ-modified RNAs were comparable to unmodified RNAs (FIG. 4). To determine whether the observed combined error rates are not dependent on the sequence of the RNA, the combined error rates in two functional mRNAs (RNA2 encoding Cypridina luciferase and RNA6 encoding part of BNT162b/Comirnaty mRNA) were analyzed. Similar to what was observed with the two synthetic sequences, error rates of 6.1±0×10⁻⁵ to 1.4±0.3×10⁻⁴ errors/base were observed for RNA2 (Cypridina luciferase) and from 4.7±0.1×10⁻⁵ to 1.3±0×10⁻⁴ errors/base for RNA6 (part of BNT162b/Comirnaty mRNA sequence) (FIG. 4). Interestingly, in presence of ψ, error/base was consistently observed to be two-fold higher than that observed with unmodified luciferase or BNT162b/Comirnaty mRNA. In contrast, consistent with RNA1 and RNA 5, when m1ψ was present in the reaction, the error/base was observed to be less than that observed with ψ-modified luciferase or BNT162b/Comirnaty mRNA (FIG. 4).

To evaluate whether the nature of the errors that are introduced when the reactions are performed with different uridine analogs is similar, the error profiles observed in the four RNA sequences were compared. Irrespective of the sequence of the RNA or the nature of the uridine analog used in the reaction, base substitution was observed to account for the predominant errors ranging from 73% to 96% of total errors (FIG. 4). To assess whether a specific substitution is more prevalent than another and if there are differences when m1ψ is present in the reaction, the substitution profile (FIG. 5) was analyzed. For unmodified RNA, no distinct substitution errors were observed for any of the sequences (FIG. 5). In contrast, a significant increase in rA→rU/dT→dA substitutions were observed when reactions were performed with either m1ψ or w (FIG. 5), likely due to m1ψTP incorporated in place of rATP (opposite dT) by T7 RNA polymerase as it is less likely that m1ψ would have a global effect on the incorporation of other bases. The rA→rU/dT→dA error rates in ψ-modified RNAs were about nine- to 12-fold higher than unmodified RNAs. The rA→rU/dT→dA substitution was also apparent (up to three-fold greater than unmodified RNAs) in m1ψ-modified RNAs but was observed to be less compared to ψ-modified counterpart (FIG. 5).

### Example 10: SP6 RNAP Incorporates m1ψ with Higher Fidelity than w; Overall Error Rates in Reactions Performed with SP6 RNAPs are Higher than Those Performed with T7 RNAP

Different RNAPs were next compared to determine if m1ψ is incorporated with varied fidelity by different ssRNAPs and if the differences observed in error rates in ψ- and m1ψ-incorporating RNAs synthesized with T7 RNAP are also observed with other ssRNAPs, T3 and SP6 RNAPs. SP6 RNAP shares 32% identity to T7 RNAP and also may be used for generating synthetic mRNAs for therapeutic applications. On the other hand, T3 RNAP is 82% identical to T7 RNAP. The total combined error rates observed in reactions performed with T3 RNAP were comparable to reactions performed with T7 RNAP. Accordingly, these two closely related RNAPs may have similar fidelity profiles (FIG. 23). On the other hand, comparison of the total combined error rates observed from modified and unmodified RNAs synthesized with SP6 RNAP demonstrated two-fold to three-fold higher error rates than those observed with T7 RNAP (FIG. 4 and FIG. 9). For unmodified RNA1/RNA5, the total combined error rates observed when reactions were performed with SP6 RNAP under standard high-yield reaction conditions were greater than that of T7 RNAP (FIG. 9) with combined error rates of 1.4±0.3×10⁻⁴ and 1.3±0.3×10⁻⁴ errors/base, respectively (FIG. 9). For ψ-modified RNA1/RNA5 and RNA2 the error rate was observed to be 3.1±0.1×10⁻⁴ and 3.4±0×10⁻⁴ errors/base, respectively. The total combined error rates of m1ψ-incorporated RNA1/RNA5 and RNA2 were both 2.5±0×10⁻⁴ errors/base. Similar to T7 RNAP, combined error rates followed the same trend-ψ-modified RNAs demonstrating highest error rates as compared to m1ψ-modified RNAs and uridine-containing RNAs. (FIG. 4 and FIG. 9).

Furthermore, base substitution errors were observed to be the most predominant error type ranging between 84% to 96% of total errors with SP6 RNAP (FIG. 9). In unmodified RNAs synthesized with SP6 RNAPs, the rA→rG/dT→dC substitution was observed to be the predominant error (FIG. 10). However, the substitution profiles of m1ψ- or ψ-modified RNA demonstrated a preponderance of rA→rU/dT→dA substitutions as observed with m1ψ- or ψ-modified RNAs synthesized with T7 RNAP (FIG. 5 and FIG. 10). Compared to the error rates of unmodified RNA, ψ-modified RNAs had seven- to nine-fold increase in rA→rU/dT→dA substitution in RNA1/RNA5 and RNA2, respectively. For the m1ψ-modified RNAs, the combined error rates for RNA1/RNA5 and RNA2 sequences were two- to three-fold higher than unmodified RNA.

### Example 11: Fidelity of Uridine Incorporation is not Dependent on the Total rNTP Concentration of In Vitro Transcription Reaction

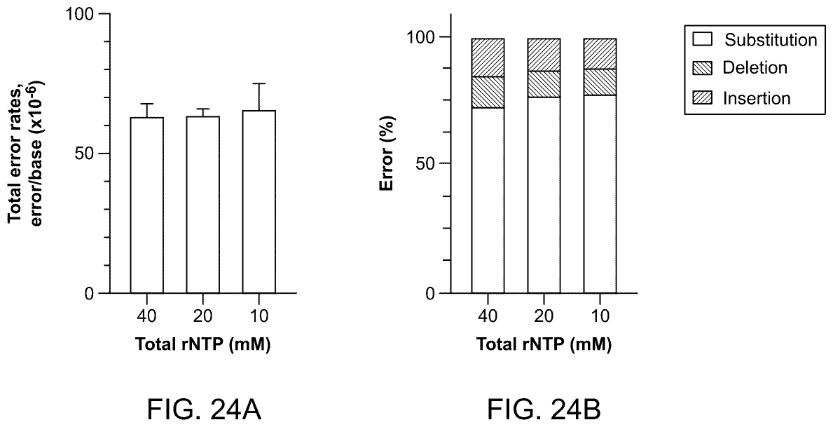

For synthetic mRNA-based applications, high yield of RNA from the in vitro transcription reaction is desirable and reactions are typically performed with high concentrations of rNTPs. The recommended high-yield rNTP concentrations are different for T7 RNAP (40 mM rNTP) and SP6 RNAP (20 mM rNTP). To assess whether the differences in combined error observed for T7 RNAP and SP6 RNAP are not due to differences in the rNTP concentrations in the reactions, in vitro transcription reactions with T7 under low rNTP reaction conditions were performed with either 20 mM or 10 mM rNTP. The total combined error as well as the base substitution errors observed in unmodified RNA1/RNA5 when reactions were performed with T7 RNAP under low rNTP (20 or 10 mM) reaction conditions were comparable to those observed with high rNTP (40 mM) reaction conditions (FIG. 24). Thus, the overall rNTP concentration in the reaction may not affect fidelity of uridine incorporation and the differences in error rates observed with T7 and SP6 RNAP may not be due to differences in the reaction conditions. Furthermore, the total combined error as well as the base substitution error profile observed in m1ψ- and ψ-modified RNAs were also unaltered under low rNTP reaction conditions.

### Example 12: Altering the rNTP Composition During In Vitro Transcription Reduces Combined Error Rate and the Predominant rA-to-rU Substitution Error

Data disclosed here demonstrates that m1ψ- and ψ-modified RNAs have increased combined errors compared to unmodified RNAs and the increased rA→rU substitution errors account for most of the misincorporations that are observed (FIG. 5 and FIG. 10). Furthermore, in view of the increased rA→rU substitution errors observed in RNAs synthesized with SP6 RNAPs the predominant rA→rU substitution might occur during in vitro transcription where the uridine analogs are misincorporated with higher frequency. To test this hypothesis as well as to reduce the rA→rU substitution during in vitro transcription reactions with uridine analogs, the rUTP concentrations in the reaction were manipulated with the idea that balancing the rUTP in the in vitro transcription reaction to be proportional to the nucleotide composition of the RNA sequence to be synthesized might result in reduced rA→rU substitution errors and consequently the total errors observed during in vitro transcription.

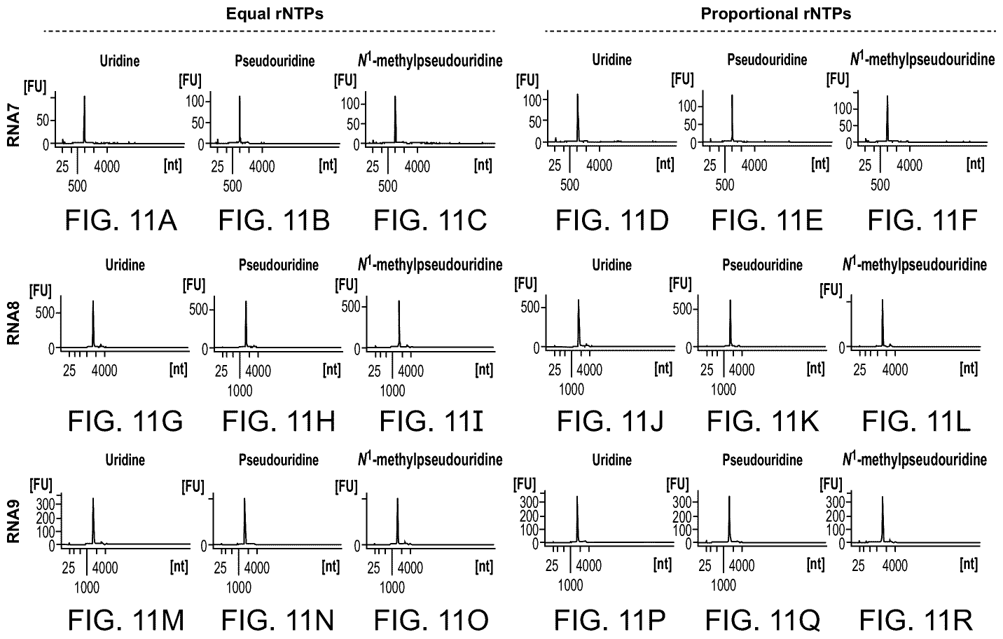

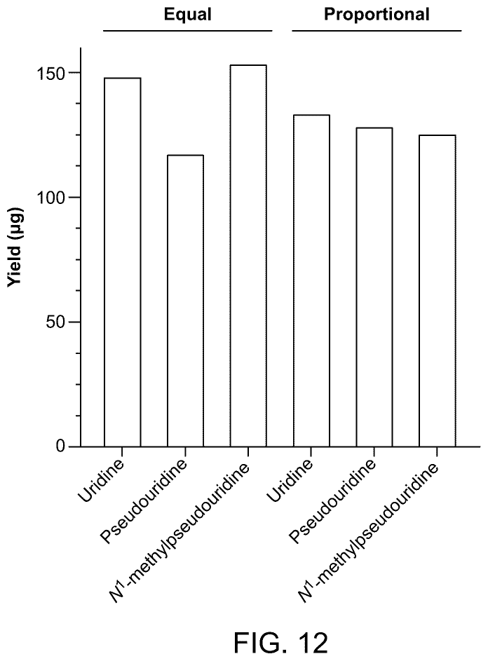

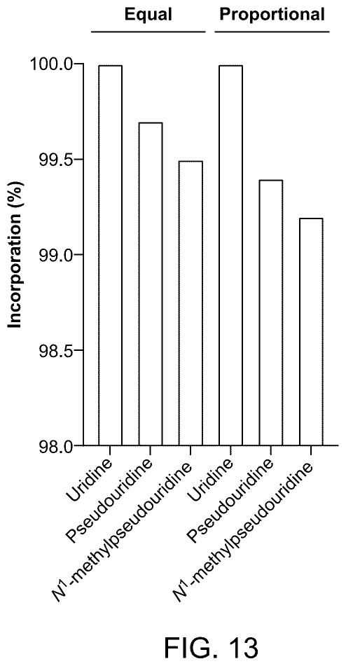

Combining sequence optimization of the synthetic RNA with incorporation of uridine modifications, specifically m1ψ and ψ, in synthetic mRNA-based vaccines and therapeutics may become a common practice. In addition, depleting the uridine content in the synthetic mRNA by sequence optimization may reduce the immunogenicity of the synthetic molecules. To test the effect of reduced rUTP in the reactions, a template for uridine-depleted randomized sequence (RNA7) was generated that yields a final RNA base composition of 30.9% A, 33.4% C, 30.2% G, and 5.5% U. First, as a control, the error rates were analyzed when in vitro transcription reactions were performed under standard high-yield rNTP condition where all the rNTPs are added equally to a final concentration of 40 mM (represented as equal in FIG. 11, FIG. 12, FIG. 13, FIG. 14, and FIG. 15). The uridine-depleted RNA7 transcribed with T7 RNAP with equal molar unmodified rNTPs had a total combined error rate 6.4±0.7×10⁻⁵ errors/base, while the error rates of ψ- and m1ψ-incorporating transcripts were observed to be 1.9±0.2×10⁻⁴ and 9.5±2.7×10⁻⁵ errors/base, respectively (FIG. 14A). For the rUTP optimized reactions, rNTPs proportional to the sequence encoded by the template sequence, i.e., 12.4 mM (30.9%) rATP, 13.2 mM (33.4%) rCTP, 12 mM (30.2%) rGTP and 2.4 mM (5. %) rUTP, were used. No difference in the RNA yield and RNA integrity from the in vitro transcription reactions were observed when the rNTP levels were altered (FIG. 11 and FIG. 12). Furthermore, the modifications were also incorporated efficiently (FIG. 13). The total combined error rate of unmodified RNA was observed to be 5.2±0.4×10⁻⁵ errors/base and that of m1ψ-incorporating RNA was 6.6±0.5×10⁻⁵ errors/base (FIG. 14A). Noticeably, the total error rate of ψ-modified RNA was 7.6±0.6×10⁻⁵ errors/base, about two-fold reduced as compared to equal molar rNTP condition (1.9±0.2×10⁻⁴ errors/base).

Under standard equal molar rNTP reaction conditions, the substitution error profile of the uridine-depleted RNA7 sequence resembled RNA1/RNA5, RNA2 and RNA6, with rA→rU/dT→dA substitution demonstrating the most significant change when modified uridine was used in the reaction. For m1ψ-modified RNA, the rA→rU/dT→dA substitution was increased three-fold as compared to the unmodified RNA and this was even more pronounced in the ψ-modified RNA with an increase of 14-fold over unmodified RNA. Interestingly, when the molar ratio of rNTPs was proportioned to the nucleotide content of the sequence to be synthesized, the rA→rU substitution error rates were lowered significantly as compared to that observed under standard reaction condition (equal rNTP conditions). For unmodified RNA, a four-fold reduction in rA→rU substitution was observed when the molar ratio of rUTP was proportioned to the nucleotide content of the sequence to be synthesized (FIG. 15A). Similarly, six- and seven-fold reductions were observed for ψ-modified and m1ψ-modified RNA, respectively. Furthermore, no significant change in any of the other substitution errors were observed when the rNTP concentrations were altered (FIG. 15A). The comparison of the two different RNA synthesis workflows with RNA7 demonstrates that lowering the molar ratio of rUTP to be proporational to the nucleotide content of the RNA to be synthesized indeed reduced the rA→rU substitution error during in vitro transcription reaction and further provides support that the rA→rU substitutions observed with the uridine modifications are errors observed during in vitro transcription. Furthermore, to assess whether the effect of altering the rNTP concentration to reduce the rA→rU substitution is observed with different sequences, two other templates for uridine-depleted randomized sequences were generated, RNA8 (29.7% A, 32.0% C, 31.7% G, and 6.6% U) and RNA9 (30.2% A, 30.4% C, 27.1% G, and 12.3% U). Similar to what was observed with RNA7, reduced rA→rU substitution errors were observed for modified and unmodified RNAs (FIG. 14B, FIG. 14C, FIG. 15B, and FIG. 15C) when the molar ratio of the rNTPs were altered to be proportional to the nucleotide content of the RNA to be synthesized.

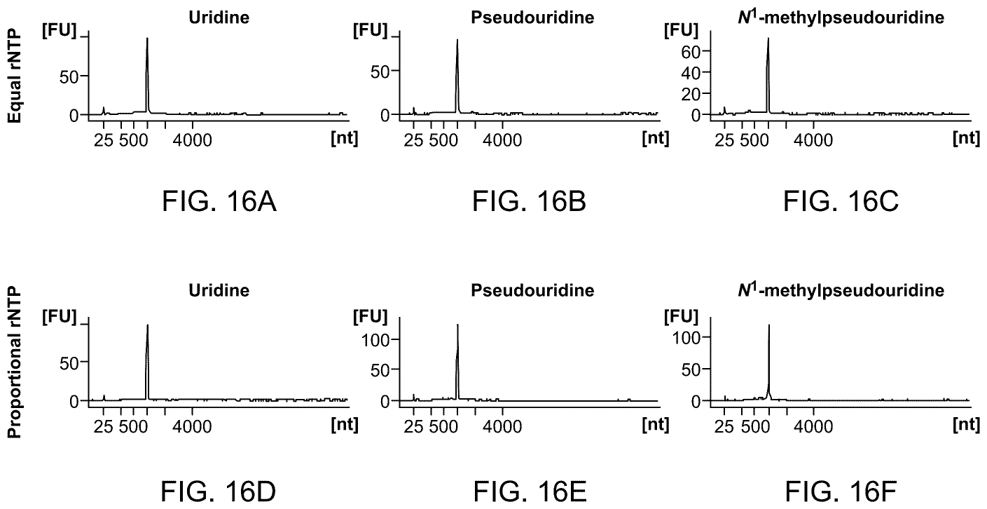

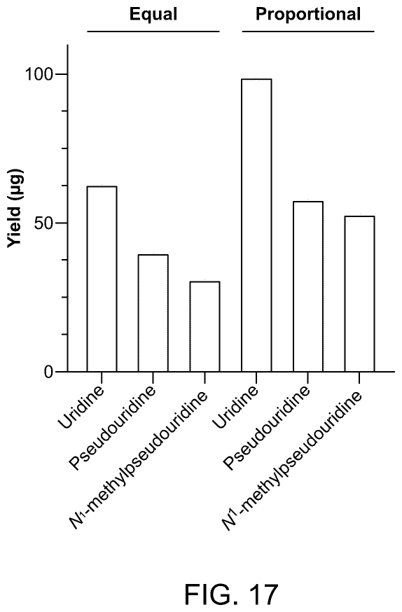

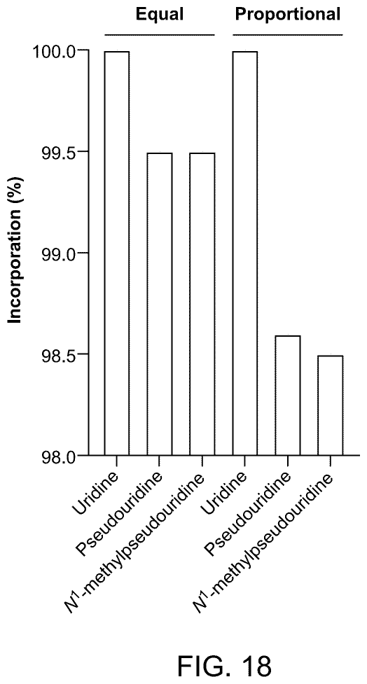

In light of the observation that the fidelity of T7 RNAP can be modulated by adjusting the rNTP ratio in the reaction, the possibility was investigated that the same holds true for SP6 RNAP where the rA→rU substitution is also the most predominant substitution error in modified RNAs (FIG. 16, FIG. 17, and FIG. 18). T-depleted randomized sequence template (RNA7) that consists of 30.9% A, 33.4% C, 30.2% G, and 5.5% U was used and reactions were performed with either equimolar rNTPs (5 mM each) or rNTPs at a molar ratio proportioned to the nucleotide sequence of the RNA to be synthesized (6.2 mM (30.9%) rATP, 6.6 mM (33.4%) rCTP, 6 mM (30.2%) rGTP and 1.2 mM (5.5%) rUTP). In presence of proportional rNTP concentrations, the total combined error rate in unmodified RNA7 was reduced to 1.3±0×10⁻⁴ errors/base, one-fold decrease from 2.8±0.9×10⁻⁴ errors/base in presence of equal molar unmodified rNTP (FIG. 19). Similarly, for m1ψ-modified reaction, the total combined error rate was reduced two-fold (from 4.1±1.0×10⁻⁴ errors/base to 1.5±0×10⁻⁴ errors/base). As observed with T7 RNAP, ψ-modified RNA had the most pronounced (three-fold) reduction in total combined error rate (from 6.0±2.1×10⁻⁴ errors/base to 1.7±0×10⁻⁴ errors/base) when the rNTP ratios were altered to be proportional to the nucleotide composition of RNA7. Furthermore, the reduction in total combined error observed in proportional rNTP reaction conditions is attributable specifically to reduction in the rA→rU substitution errors (FIG. 20).

U-depletion of the RNA sequence may not be a viable alternative for all sequences. For example, the extent of U-depletion may be dependent on the sequence since changes in the U-content may have to be balanced with a need or desire to avoid altering the codon. A corollary approach was investigated in which increasing the rATP concentrations in the reaction might also reduce the rA→rU substitutions in the in vitro transcription reactions. In vitro transcription of RNA1/RNA5 was performed with excess rATPs (20 mM rATP with 10 mM of other rNTPs or 16 mM rATP with 8 mM of other rNTPs). When 20 mM rATP was used in the reactions, the total combined error rates of unmodified, ψ- and m1ψ-modified RNA1/RNA5 were observed to be 4.3±0.1×10⁻⁵ errors/base, 5.1±0.1×10⁻⁵ errors/base and 3.4±0.1×10⁻⁵ errors/base, respectively (FIG. 21). A two-fold decrease in total combined error rates was observed for m1ψ-modified and ψ-modified RNAs and a 1.5-fold for unmodified RNAs (FIG. 21) when the rATP concentration was increased with respect to the other rNTPs. As hypothesized, the substitution error profile of reactions performed with excess rATP showed rA→rU/dT→dA substitution in the modified RNA changed the most compared to unmodified RNA (FIG. 22).

Taken together, these results demonstrate that the rA→rU substitution that is observed when in vitro transcription reactions are performed with uridine analogs, stems from substitution errors during in vitro transcription with T7 and SP6 RNAP. Furthermore, the fidelity of the uridine analog incorporation can be increased by either lowering the molar ratio of rUTP to be proportional to the nucleotide composition of the synthetic RNA sequence or increasing the molar ratio of rATP in the reaction without compromising the yield from the reaction.

### Example 13: Fidelity of KP34 RNAP Incorporation of Unmodified and Modified Uridine

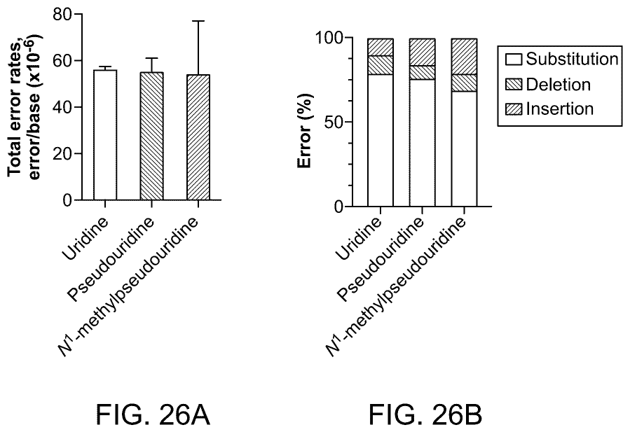

To further characterize RNAPs with distinct primary protein sequence, in vitro transcription reactions with KP34 RNAP were performed. KP34 RNAP's overall error rate for modified uridine incorporation is lower than T7 RNAP and SP6 RNAP. KP34 RNAP shares 28% sequence identity to T7 RNAP and 26% to SP6 RNAP. The total combined error rate of uridine incorporation observed in the reaction performed with KP34 RNAP is 56±1×10⁻⁶ errors/base, which is comparable to that of T7 RNAP and two-fold less than SP6 RNAP (FIG. 4, FIG. 9 and FIG. 26). The total combined error rates of w incorporation observed in the reaction performed with KP34 RNAP is 55±6×10⁻⁶ errors/base, two-fold less than that of T7 RNAP and six-fold less than SP6 RNAP (FIG. 4, FIG. 9 and FIG. 26). The total combined error rates of m1ψincorporation observed in the reaction performed with KP34 RNAP is 54±23×10⁻⁶ errors/base, comparable to that of T7 RNAP and five-fold less than SP6 RNAP (FIG. 4, FIG. 9 and FIG. 26). Taken together, KP34 RNAP, unlike T7 RNAP and SP6 RNAP, incorporates modified nucleotides with less perturbed fidelity, suggesting that a unique active site in KP34 RNAP can position the correct incoming ψTP and m1ψTP.

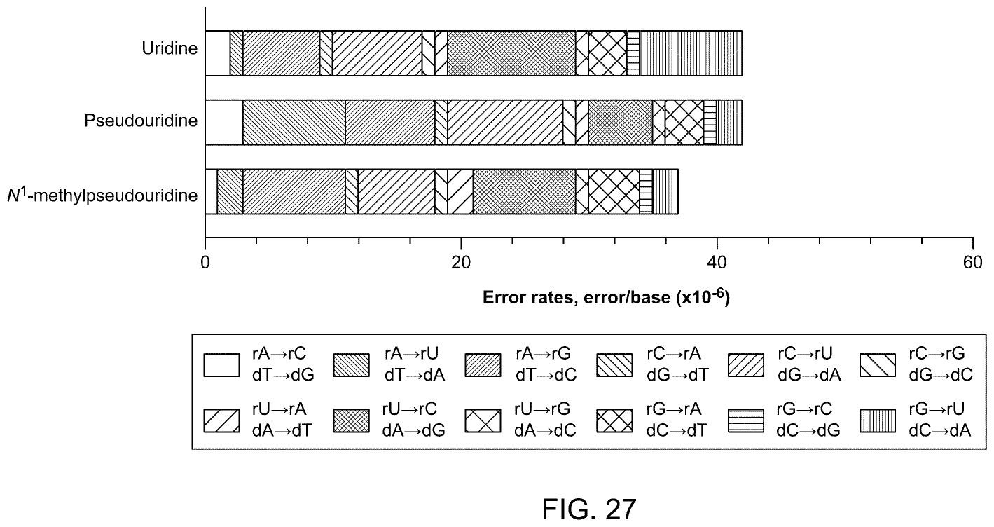

The most predominant error type with KP34 RNAP is substitution, ranging from 69% to 79% (FIG. 25). In the substitution error profile of unmodified RNA1/RNA5 synthesized with KP34 RNAP, the rU→rC/dA→dG substitution was the predominant error (FIG. 27). The substitution profile of ψ-modified RNA1/RNA5 with KP34 RNAP demonstrated a preponderance of rC→rU/dG→dA, while that of m1ψ-modified RNA1/RNA5 showed a preponderance of rA→rG/dT→dC and rU→rC/dA→dG (FIG. 27). The substitution profiles of ψ-modified and m1ψ-modified RNA with KP34 RNAP are distinct from those with T7 RNAP or SP6 RNAP, where rA→rU/dT→dA is the major substitution.

### Example 14: Hi-T7 RNAP Incorporates m1 □ with Higher Fidelity than □ Regardless of Temperatures; Overall Error Rates in the Reaction Performed with Hi-T7 RNAP are Comparable to T7 RNAP

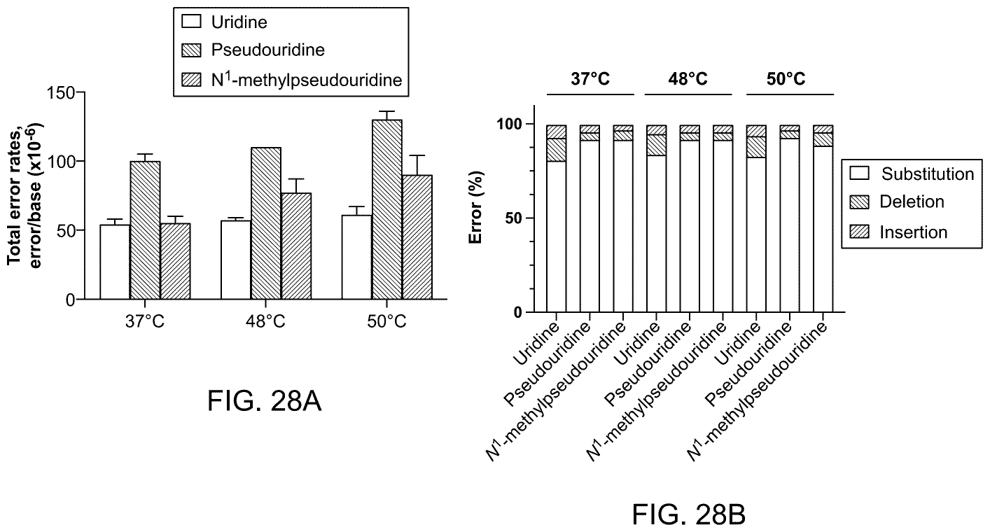

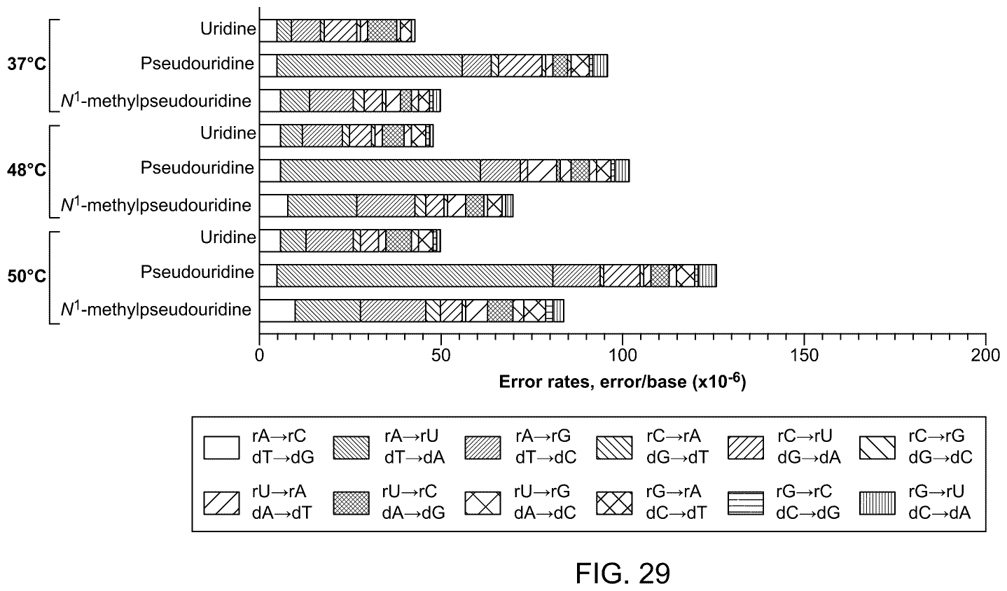

Synthetic mRNA made with Hi-T7 RNAP, a thermostable RNAP that is engineered from T7 RNAP, at elevated temperatures has been shown to generate less dsRNA byproducts. The use of Hi-T7 RNAP can simplify the process of in vitro mRNA production as less purification step is required. To determine if high temperatures have any impacts on the fidelity of in vitro transcription, reactions with Hi-T7 RNAP were performed at 37° C., 48° C. and 50° C. The total combined error rates with Hi-T7 RNAP to incorporate unmodified and modified nucleotides at the three temperatures tested were comparable to T7 RNAP at 37° C. (FIG. 4 and FIG. 28). There was a slight increase in total error rates at higher temperature. Hi-T7 RNAP also exhibited the same trends of fidelity-U>m1ψ>ψ. The predominant error type is substitution, ranging from 81% to 92%. The substitution error profile of unmodified RNA1/RNA5 with Hi-T7 RNAP showed predominant error is is rC→rU/dG→dA at 37° C., and rA→rG/dT→dC at both 48° C. and 50° C. (FIG. 29). Similar to T7 RNAP, the predominant error of ψ-modified RNA1/RNA5 is rA→rU/dT→dA when the reactions were performed with Hi-T7 RNAP at all three temperatures tested (FIG. 29). The predominant error rA→rU/dT→dA was also observed in the substitute error profile of m1ψ-modified RNA1/RNA5 at all three temperatures but reduced by three-fold to six-fold compared to ψ-modified RNA1/RNA5. Together, reactions at temperature higher than 10° C. lead to minimal increase in total error rates, and no noticeable change in the substitution error profiles.

TABLE 1
Sequences
Template sequences used in this study. Underlined sequence denotes T7
promoter; Underlined bold sequence denotes restriction enzyme sites used
for template linearization (HpaI for RNA1, RNA5, RNA7, RNA8 and RNA9;
NotI for RNA2; NheI for RNA6).

| | | SEQ ID |
|---|---|---|
| Name | Sequence | NO: |
| RNA1 | TAATACGACTCACTATAGGGTCTAGAAATAATTTTGTTTA | 1 |
| | ACTTTAGAGTACACGAGTCAGGCTACAGCATCCTCTGGT | |
| | TCAGACTACTTGATTCATGTGTACCCTATATGCGAGGAT | |
| | ATGTGTATCGTAGAAATTGTCAGGCAGTAACGTTCCGCG | |
| | AGTTTTAATGGGCGCGCCATGACTCTAAGAGTGATATAC | |
| | CTCCTCGGTCTCGGGCCCGGGGTGTAATTAGCCCAGTTA | |
| | GACACGATCGCCCGACGTATATTGTTGCTTGGGTATCGT | |
| | CGCATGCGAAGTATTGCCCAAGGAGACACAACAAGCAA | |
| | CTTATGTTGACTCCCTTCGACCATTAAAATTTGTTAGAAC | |
| | GGACAGAAAGGATGCGCCTTATAAATGTCCTGTGCAGTG | |
| | ATGAAGCGACCTCAAAACGCTTCATGATCTAACCGACTC | |
| | ACCTTGCCGTTCCCTCCGCGCCTTAAAACCGGCCGGTCTT | |
| | GCGAAAAGCGGGAAACGAGTTTACCCACGGATAGCAGG | |
| | GAATGTTGCGGCTGGCTAGGGAGCATGAAGGTAGATACT | |
| | CCACGGCTTACCTTTCCGGGGCTCAACATCTAGCCACAG | |
| | ACCTTTTCGTTAAGCCCACCCCCACTGGATACTGAATCAT | |
| | CAGGGAACCGGACCCAACCAGTTTGGGCTCGTCCAAGCT | |
| | TCGGTCTCGTCCCTAAGTGCAAAGATATGGAAAGAGCAG | |
| | CATAGGTATATGGATTATTCTTTTACCACTCGTTTCTTAC | |
| | CGTAACTTACGCAATGGATCACGTGCCGAGGCGGCGGTA | |
| | CAGCTGTTCGAAGGGCTCTGTCGGGAACGCTAACATCCA | |
| | GCCGGTAAATTCCAAACTAGGGAAAGGACACGCACTGA | |
| | ATTGAATATAGTCGTGAAGGGTGGTGTAAGTCGTGCACA | |
| | GCCCGCATTAAGTACTAAACAGCGTCCAATCTTGATCTA | |
| | CTTACGGCCTGATGTTCTTCAGCACCTCCTAGCACTGGA | |
| | GTACTTCGCTATCAATGAGATTAGCACTTTGTACATGTCA | |
| | TCCAGCCCGAGTCTGGGGTCCGACAATGCGGTCGCCGAT | |
| | TGGTATCTGCATGTAGTATTAAACGGAGCTGCCGCGGCT | |
| | GCGGATTATAGTTCATGTCTTGACGGTCCTCGTGAACTGT | |
| | GGTTAAC | |
| RNA2 | TAATACGACTCACTATAGGGAGACCCAAGCTTGGTACCG | 2 |
| | AGCTCGGATCCGCCACCATGAAGACCTTAATTCTTGCCGT | |
| | TGCATTAGTCTACTGCGCCACTGTTCATTGCCAGGACTGT | |
| | CCTTACGAACCTGATCCACCAAACACAGTTCCAACTTCCT | |
| | GTGAAGCTAAAGAAGGAGAATGTATTGATAGCAGCTGTG | |
| | GCACCTGCACGAGAGACATACTATCAGATGGACTGTGTG | |
| | AAAATAAACCAGGAAAAACATGTTGCCGAATGTGTCAGT | |
| | ATGTAATTGAATGCAGAGTAGAGGCCGCAGGATGGTTTA | |
| | GAACATTCTATGGAAAGAGATTCCAGTTCCAGGAACCTG | |
| | GTACATACGTGTTGGGTCAAGGAACCAAGGGCGGCGACT | |
| | GGAAGGTGTCCATCACCCTGGAGAACCTGGATGGAACCA | |
| | AGGGGGCTGTGCTGACCAAGACAAGACTGGAAGTGGCTG | |
| | GAGACATCATTGACATCGCTCAAGCTACTGAGAATCCCA | |
| | TCACTGTAAACGGTGGAGCTGACCCTATCATCGCCAACCC | |
| | GTACACCATCGGCGAGGTCACCATCGCTGTTGTTGAGATG | |
| | CCAGGCTTCAACATCACCGTCATTGAGTTCTTCAAACTGA | |
| | TCGTGATCGACATCCTCGGAGGAAGATCTGTAAGAATCG | |
| | CCCCAGACACAGCAAACAAAGGAATGATCTCTGGCCTCT | |
| | GTGGAGATCTTAAAATGATGGAAGATACAGACTTCACTT | |
| | CAGATCCAGAACAACTCGCTATTCAGCCTAAGATCAACC | |
| | AGGAGTTTGACGGTTGTCCACTCTATGGAAATCCTGATGA | |
| | CGTTGCATACTGCAAAGGTCTTCTGGAGCCGTACAAGGA | |
| | CAGCTGCCGCAACCCCATCAACTTCTACTACTACACCATC | |
| | TCCTGCGCCTTCGCCCGCTGTATGGGTGGAGACGAGCGA | |
| | GCCTCACACGTGCTGCTTGACTACAGGGAGACGTGCGCT | |
| | GCTCCCGAAACTAGAGGAACCTGCGTTTTGTCTGGACATA | |
| | CTTTCTACGATACATTTGACAAAGCAAGATACCAATTCCA | |
| | GGGTCCCTGCAAGGAGATTCTTATGGCCGCCGACTGTTTC | |
| | TGGAACACTTGGGATGTGAAGGTTTCACACAGGAATGTT | |
| | GACTCTTACACTGAAGTAGAGAAAGTACGAATCAGGAAA | |
| | CAATCGACTGTAGTAGAACTCATTGTTGATGGAAAACAG | |
| | ATTCTGGTTGGAGGAGAAGCCGTGTCCGTCCCGTACAGCT | |
| | CTCAGAACACTTCCATCTACTGGCAAGATGGTGACATACT | |
| | GACTACAGCCATCCTACCTGAAGCTCTGGTGGTCAAGTTC | |
| | AACTTCAAGCAACTGCTCGTCGTACATATTAGAGATCCAT | |
| | TCGATGGTAAGACTTGCGGTATTTGCGGTAACTACAACCA | |
| | GGATTTCAGTGATGATTCTTTTGATGCTGAAGGAGCCTGT | |
| | GATCTGACCCCCAACCCACCGGGATGCACCGAAGAACAG | |
| | AAACCTGAAGCTGAACGACTCTGCAATAGTCTCTTCGCCG | |
| | GTCAAAGTGATCTTGATCAGAAATGTAACGTGTGCCACA | |
| | AGCCTGACCGTGTCGAACGATGCATGTACGAGTATTGCCT | |
| | GAGGGGACAACAGGGTTTCTGTGACCACGCATGGGAGTT | |
| | CAAGAAAGAATGCTACATAAAGCATGGAGACACCCTAGA | |
| | AGTACCAGATGAATGCAAATAGGCGGCCGC | |
| RNA5 | TAATACGACTCACTATAGGGTCTAGAAATAATTTTGTTTA | 3 |
| | ACTTTAGAGTACACGAGTCAGGCTACAGCATCTTGACAC | |
| | CAGAATATTATGGATTGGACGCTTCCCACTAAATGGAAG | |
| | ACTGTTCGGTCATAAACACTACTAGGAATTCCTCTCCAGT | |
| | CATCATGTTCGATCGTCTAGCAGCAATCTCTTCCGATCGA | |
| | TATTTGCGCGTGACTCAGGCGAGCCCATGACAGCTTCTCC | |
| | CCGTGAGAACCACGACTAGAAGTTATCTGTTGAGCTGCT | |
| | AGCTTCGTGGCCCGGCCATGGTAGTAGCGGCTCACTCGC | |
| | GCTAACTTTGCCTGCTCGAGAAAACGGGCGAAACACCCA | |
| | GCAACACAAGCCACTTAATTTGTTGATAGATAATAAGAT | |
| | CAGGTTATTAGTCGCTCTGCACTTACTTTAAGTGCCAACT | |
| | ATGCTGTATCGGCCAGGGTGAAAACGGGTGCCGCCACTIC | |
| | aGTGTGTCGGAGTCTGCTGACGGATTAGGGCACAGACGTA | |
| | TGGTTATATCCTAAGGTAGTGTGTCAATGTACTGGGGACA | |
| | AAGTCAGTGGGCACCGCATCAGGAGTGCAACCTCCGCTA | |
| | GTACCGACTCGTCAATGCTTTGAGCGATGGCTTGCGCTCC | |
| | CAAATCCTTAAGCTTTTATGCATTCGGCTCTGGCCCTCAG | |
| | GCCTGACCTGGAATTTCATCGGAAACGCCTTAACCGACAT | |
| | TACATCGACACCAAGATCCCGACGCTTCATGCGGAGACG | |
| | ATAGAGACTCTAACCAAGAATAAAAGGAGTAGTCCCTAA | |
| | TCTACTGAAACGGGGATACCTCAAATCACGGGAATGCGT | |
| | TACTGACCCGCTATGTGAGGCTCGGATCACCCTCGTTCTA | |
| | TTGCCTTGTAATCATGGTGGGGCGGCGGAGCGGGATTAG | |
| | AGGGTGTCCCTAATGTGAGTAGATCTGTAGTAATGATAC | |
| | GTCTCCTCAATATGAGGCGTATTGCAGGTCACAGCACAG | |
| | GGAGATTTCGGCGCACCCAGCCGAGTTGCCTCCGTCGTTG | |
| | TTTAGGTATATGCATAACTGCTCACGACAAATACAGCAG | |
| | AGCCTACGTTGGGTTATCGAATCCTTGTGGACAAGAAGCT | |
| | TCTTCATGTCTTGACGGTCCTCGTGAACTGTGGTTAAC | |
| RNA6 | TAATACGACTCACTATAGGAGAATAAACTAGTATTCTTCT | 4 |
| | GGTCCCCACAGACTCAGAGAGAACCCGCCACCATGTTCG | |
| | TGTTCCTGGTGCTGCTGCCTCTGGTGTCCAGCCAGTGTGT | |
| | GAACCTGACCACCAGAACACAGCTGCCTCCAGCCTACAC | |
| | CAACAGCTTTACCAGAGGCGTGTACTACCCCGACAAGGT | |
| | GTTCAGATCCAGCGTGCTGCACTCTACCCAGGACCTGTTC | |
| | CTGCCTTTCTTCAGCAACGTGACCTGGTTCCACGCCATCC | |
| | ACGTGTCCGGCACCAATGGCACCAAGAGATTCGACAACC | |
| | CCGTGCTGCCCTTCAACGACGGGGTGTACTTTGCCAGCAC | |
| | CGAGAAGTCCAACATCATCAGAGGCTGGATCTTCGGCAC | |
| | CACACTGGACAGCAAGACCCAGAGCCTGCTGATCGTGAA | |
| | CAACGCCACCAACGTGGTCATCAAAGTGTGCGAGTTCCA | |
| | GTTCTGCAACGACCCCTTCCTGGGCGTCTACTACCACAAG | |
| | AACAACAAGAGCTGGATGGAAAGCGAGTTCCGGGTGTAC | |
| | AGCAGCGCCAACAACTGCACCTTCGAGTACGTGTCCCAG | |
| | CCTTTCCTGATGGACCTGGAAGGCAAGCAGGGCAACTTC | |
| | AAGAACCTGCGCGAGTTCGTGTTTAAGAACATCGACGGC | |
| | TACTTCAAGATCTACAGCAAGCACACCCCTATCAACCTCG | |
| | TGCGGGATCTGCCTCAGGGCTTCTCTGCTCTGGAACCCCT | |
| | GGTGGATCTGCCCATCGGCATCAACATCACCCGGTTTCAG | |
| | ACACTGCTGGCCCTGCACAGAAGCTACCTGACACCTGGC | |
| | GATAGCAGCAGCGGATGGACAGCTGGTGCCGCCGCTTAC | |
| | TATGTGGGCTACCTGCAGCCTAGAACCTTCCTGCTGAAGT | |
| | ACAACGAGAACGGCACCATCACCGACGCCGTGGATTGTG | |
| | CTCTGGATCCTCTGAGCGAGACAAAGTGCACCCTGAAGT | |
| | CCTTCACCGTGGAAAAGGGCATCTACCAGACCAGCAACT | |
| | TCCGGGTGCAGCCCACCGAATCCATCGTGCGGTTCCCCAA | |
| | TATCACCAATCTGTGCCCCTTCGGCGAGGTGTTCAATGCC | |
| | ACCAGATTCGCCTCTGTGTACGCCTGGAACCGGAAGCGG | |
| | ATCAGCAATTGCGTGGCCGACTACTCCGTGCTGTACAACT | |
| | CCGCCAGCTTCAGCACCTTCAAGTGCTACGGCGTGTCCCC | |
| | TACCAAGCTGAACGACCTGTGCTTCACAAACGTGTACGC | |
| | CGACAGCTTCGTGATCCGGGGAGATGAAGTGCGGCAGAT | |
| | TGCCCCTGGACAGACAGGCAAGATCGCCGACTACAACTA | |
| | CAAGCTGCCCGACGACTTCACCGGCTGTGTGATTGCCTGG | |
| | AACAGCAACAACCTGGACTCCAAAGTCGGCGGCAACTAC | |
| | AATTACCTGTACCGGCTGTTCCGGAAGTCCAATCTGAAGC | |
| | CCTTCGAGCGGGACATCTCCACCGAGATCTATCAGGCCG | |
| | GCAGCACCCCTTGTAACGGCGTGGAAGGCTTCAACTGCT | |
| | ACTTCCCACTGCAGTCCTACGGCTTTCAGCCCACAAATGG | |
| | CGTGGGCTATCAGCCCTACAGAGTGGTGGTGCTGAGCTTC | |
| | GAACTGCTGCATGCCCCTGCCACAGTGTGCGGCCCTAAG | |
| | AAAAGCACCAATCTCGTGAAGAACAAATGCGTGAACTTC | |
| | AACTTCAACGGCCTGACCGGCACCGGCGTGCTGACAGAG | |
| | AGCAACAAGAAGTTCCTGCCATTCCAGCAGTTTGGCCGG | |
| | GATATCGCCGATACCACAGACGCCGTTAGAGATCCCCAG | |
| | ACACTGGAAATCCTGGACATCACCCCTTGCAGCTTCGGCG | |
| | GAGTGTCTGTGATCACCCCTGGCACCAACACCAGCAATC | |
| | AGGTGGCAGTGCTGTACCAGGACGTGAACTGTACCGAAG | |
| | TGCCCGTGGCCATTCACGCCGATCAGCTGACACCTACATG | |
| | GCGGGTGTACTCCACCGGCAGCAATGTGTTTCAGACCAG | |
| | AGCCGGCTGTCTGATCGGAGCCGAGCACGTGAACAATAG | |
| | CTACGAGTGCGACATCCCCATCGGCGCTGGAATCTGCGC | |
| | CAGCTACCAGACACAGACAAACAGCCCTCGGAGAGCCAG | |
| | AAGCGTGGCCAGCCAGAGCATCATTGCCTACACAATGTC | |
| | TCTGGGCGCCGAGAACAGCGTGGCCTACTCCAACAACTC | |
| | TATCGCTATCCCCACCAACTTCACCATCAGCGTGACCACA | |
| | GAGATCCTGCCTGTGTCCATGACCAAGACCAGCGTGGAC | |
| | TGCACCATGTACATCTGCGGCGATTCCACCGAGTGCTCCA | |
| | ACCTGCTGCTGCAGTACGGCAGCTTCTGCACCCAGCTGAA | |
| | TAGAGCCCTGACAGGGATCGCCGTGGAACAGGACAAGAA | |
| | CACCCAAGAGGTGTTCGCCCAAGTGAAGCAGATCTACAA | |
| | GACCCCTCCTATCAAGGACTTCGGCGGCTTCAATTTCAGC | |
| | CAGATTCTGCCCGATCCTAGCAAGCCCAGCAAGCGGAGC | |
| | TTCATCGAGGACCTGCTGTTCAACAAAGTGACACTGGCC | |
| | GACGCCGGCTTCATCAAGCAGTATGGCGATTGTCTGGGC | |
| | GACATTGCCGCCAGGGATCTGATTTGCGCCCAGAAGTTTA | |
| | ACGGACTGACAGTGCTGCCTCCTCTGCTGACCGATGAGAT | |
| | GATCGCCCAGTACACATCTGCCCTGCTGGCCGGCACAATC | |
| | ACAAGCGGCTGGACATTTGGAGCAGGCGCCGCTCTGCAG | |
| | ATCCCCTTTGCTATGCAGATGGCCTACCGGTTCAACGGCA | |
| | TCGGAGTGACCCAGAATGTGCTGTACGAGAACCAGAAGC | |
| | TGATCGCCAACCAGTTCAACAGCGCCATCGGCAAGATCC | |
| | AGGACAGCCTGAGCAGCACAGCAAGCGCCCTGGGAAAG | |
| | CTGCAGGACGTGGTCAACCAGAATGCCCAGGCACTGAAC | |
| | ACCCTGGTCAAGCAGCTGTCCTCCAACTTCGGCGCCATCA | |
| | GCTCTGTGCTGAACGATATCCTGAGCAGACTGGACCCTCC | |
| | TGAGGCCGAGGTGCAGATCGACAGACTGATCACAGGCAG | |
| | ACTGCAGAGCCTCCAGACATACGTGACCCAGCAGCTGAT | |
| | CAGAGCCGCCGAGATTAGAGCCTCTGCCAATCTGGCCGC | |
| | CACCAAGATGTCTGAGTGTGTGCTGGGCCAGAGCAAGAG | |
| | AGTGGACTTTTGCGGCAAGGGCTACCACCTGATGAGCTTC | |
| | CCTCAGTCTGCCCCTCACGGCGTGGTGTTTCTGCACGTGA | |
| | CATATGTGCCCGCTCAAGAGAAGAATTTCACCACCGCTCC | |
| | AGCCATCTGCCACGACGGCAAAGCCCACTTTCCTAGAGA | |
| | AGGCGTGTTCGTGTCCAACGGCACCCATTGGTTCGTGACA | |
| | CAGCGGAACTTCTACGAGCCCCAGATCATCACCACCGAC | |
| | AACACCTTCGTGTCTGGCAACTGCGACGTCGTGATCGGCA | |
| | TTGTGAACAATACCGTGTACGACCCTCTGCAGCCCGAGCT | |
| | GGACAGCTTCAAAGAGGAACTGGACAAGTACTTTAAGAA | |
| | CCACACAAGCCCCGACGTGGACCTGGGCGATATCAGCGG | |
| | AATCAATGCCAGCGTCGTGAACATCCAGAAAGAGATCGA | |
| | CCGGCTGAACGAGGTGGCCAAGAATCTGAACGAGAGCCT | |
| | GATCGACCTGCAAGAACTGGGGAAGTACGAGCAGTACAT | |
| | CAAGTGGCCCTGGTACATCTGGCTGGGCTTTATCGCCGGA | |
| | CTGATTGCCATCGTGATGGTCACAATCATGCTGTGTTGCA | |
| | TGACCAGCTGCTGTAGCTGCCTGAAGGGCTGTTGTAGCTG | |
| | TGGCAGCTGCTGCAAGTTCGACGAGGACGATTCTGAGCC | |
| | CGTGCTGAAGGGCGTGAAACTGCACTACACATGATGACT | |
| | CGAGCTGGTACTGCATGCACGCAATCCTAGCTGCCCCTTT | |
| | CCCGTCCTGGGTACCCCGAGTCTCCCCCGACCTCGGGTCC | |
| | CAGGTATGCTCCCACCTCCACCTGCCCCACTCACCACCTC | |
| | TGCTAGTTCCAGACACCTCCCAAGCACGCAGCAATGCAG | |
| | CTCAAAACGCTTAGCCTAGCCACACCCCCACGGGAAACA | |
| | GCAGTGATTAACCTTTAGCAATAAACGAAAGTTTAACTA | |
| | AGCTATACTAACCCCAGGGTTGGTCAATTTCGTGCCAGCC | |
| | ACACCCTGGACCTAGCGCGGCCGGCTAGC | |
| RNA7 | TAATACGACTCACTATAGGGTCTAGAGCGTCGGCAAGCC | 5 |
| | ACCTAGAACAACCACGGCACGCGAACCAACAGCGGCAA | |
| | CGCCGAACGGCCACGCGACGAACTGAGCACCACCACAAG | |
| | ACCAAGCCAACGAGGAACACCGAACAACCGGTCACTGAA | |
| | CGCAACGGCCTAGGCGCGCACACCAATAAGACGGATCAA | |
| | GCCAGCGCATGACTGCGCCGGACAACGCCACGGACGACG | |
| | AACAGGTCCACCGGAGTACGACTATGCCATACCAGCAGG | |
| | ACAGCGGAGGCGACCAACAACAACAGGAGCGGCAGCCG | |
| | GCAGACGAATTGGACCGACCGGACATGGCCACATGCGAT | |
| | TGAGCCGGAACCGCGCGCACTTGACAGTCAGCAACCGGC | |
| | TCCAACAGTCCGGCACCATGAGAGAACCGGCAAGCCGGC | |
| | CTTGCCACAGGAACGCAGAGAGCGGAGCAACACAAGCC | |
| | GACGCCAAGCCAACCGCGAACCAGCCAGGCGCCGCCGGC | |
| | AACAGGAAGGTCGCACGAGCCAACATATGGACCGGCCGG | |
| | AACACTAAGGACCAAGAGGCCACTGGCGCGGAACGGAG | |
| | AACCAGGCACCAAGCAAGGAACAGAGAGGCATAGGAAC | |
| | CGAAGGCGCCATAGCCGGCGAGACGACCAGGAACGAAC | |
| | TGACAGCCAACGCTAGAGGAACGTGACGATGGACGAACG | |
| | TAGACACGGCGGCGCAACGCCGCGGCGGAGTCCGGAACC | |
| | GGCGCTAAGGTCCACACGGCAGAACCGGCCAGGCCGAGG | |
| | CAACCGCCGCCACACGACCACAGCAGCCAGAAGGATAGC | |
| | GCGCGGCCATAACAGGAACAGCAAGGACAGGCGCCACG | |
| | CAGGACCGCAACTGAGCAACCGGCAACCACCTCCACCGA | |
| | AGCCGCGAAGCAGAGGCCGACGACGGCACACGCGCCAT | |
| | GAGCGGCACAACCAGCCAGAAGGCCAACGCGCCAAGGC | |
| | GACCGAAGCCAACTACAGAAGTGTCCAAGCCAACGAAGC | |
| | CAACCGGCAGAAGAGAGCAGGGTTAAC | |
| RNA8 | TAATACGACTCACTATAGGGTCTAGAAATAATTTTGTTTA | 6 |
| | ACTTTAGAGTACACGAGTCAGGCTACAGCATCCTCCAGG | |
| | CAGCCACACAACGGAGGCAAGAGCGGCAAGCGCAACAA | |
| | CGCGCTCCGCGACGAGCGCGCAGAGTATCGGCAGATTCC | |
| | ACCGGACGGAGCACGAGGACGAAGCGACCGGTCCACAC | |
| | GGCTGGAGGACGCAGCGCCAAGCTCGGTGCCGACCGAAG | |
| | CCGAGCGAGAGGAACGCCGAGAAGCAAGCCGCGACTCC | |
| | ACAGAGCCGCCAGCCGAAGAAGACGCACGCCAGGAGGA | |
| | ACAGGAAGCGAGCGGAAGGTCAACGCACCAAGGCCAGC | |
| | AGGACTAAGCCGGCAGACGGCGGCCGCCACCACCAAGC | |
| | GAACAATGATACACGAGAACGGACCGGCAACATGGACA | |
| | CGGCGTAGTCCACAGAGCCACAAGGCAGTGGTGCGAACC | |
| | GGCAACGGAGAACAATACCGGAAGAGCACAACAGAGAG | |
| | GAGCTGGCCGGCACACGAGCGCGACGCGGCACCGGCGC | |
| | GCCACACCAGCCGAAGGCAGCCAGCACCAACGAGCGCG | |
| | GCGCACCGACTGACGGCGAGAACAGACCGAAGGCCGGC | |
| | GGCCACCACGGACGCGGACCAGAAGACCGGATGGCAGG | |
| | CGACAACGACGGCTCGCCTCCAACAAGAAGCGGCGGACC | |
| | GGCGGAAGCGACAGGCGCCGCACGTGAACACAACAACT | |
| | GCACGGCATGCGGCGAACGGCCAAGCGAGACGACGCGCT | |
| | CCAAGAGCCAACGATAGAATGCTACGGCAACCGAGCAGA | |
| | GGCAGAGCGCCAGCGCAACACACCAGAAGAACGCGACG | |
| | AGAGAGACGGACGCCGGCGCGGACCATTGGAGGCGGAG | |
| | AACCGACAACCGCAATGCGGAGATCAAGGCGGACCGAA | |
| | CGACAACAGCAGAGCCACCAACTGCGCCGTGGTCGCAAC | |
| | CAACCTAACCAGCAACAACAGGCACCGCCGCGGCGACAC | |
| | AGGACGAGGCCGCCGAACAACGATAACGCGACACCAGG | |
| | CACACACACGAACCGCGGAAGGACCACAGCGACAGCCA | |
| | TTCATGTCTTGACGGTCCTCGTGAACTGTGGTTAAC | |
| RNA9 | TAATACGACTCACTATAGGGTCTAGAAATAATTTTGTTTA | 7 |
| | ACTTTAGAGTACACGAGTCAGGCTACAGCATCCTCACAC | |
| | ACCAATGATCCAATAACCTCGAAGGCGACACCGCATGTC | |
| | GCCACCAGCCGTGAACGACGGAAGAATAACCGCAGCGA | |
| | ATACGGACATCAAGAGGCACAGAACCTACGAAGCAACAC | |
| | GAACGCACACCATAACTTCAGCGGCAACGAAGCCGACTG | |
| | AGGTCCGGCCGGCAACCAAGGCCGAAGTTCACTAAGCCT | |
| | CGTAACGGCAGCCGTATAGGACCTCGCCGAGCACCAGAC | |
| | GACACCAAGAGAAGGCAGCGACCAACTGCGTCGGCCGGC | |
| | GAACTGCCGAAGCTAAGGCACAGACCGGTCGCCAGGCCG | |
| | CAGACCGCGGAACCAACAAGAGGCAGCGCTAGGCCATCT | |
| | CAGCACCACCAAGACATTGATATTAACAGAACGCGGAAT | |
| | CAAGCGTACGATACCAACGACAAGTGTGACGAACGTGCG | |
| | TCTGAATGGCACATAAGGTCGCCGGTAGCGAACGCCGAA | |
| | CCGAACCACCTACCGCAACCAACCGACACCATAGGATGT | |
| | CACAGACGACTCGCAACGGCTAGACCTTACCAACACAGG | |
| | AACATCACAAGAATCATAACACGAGCCACAAGAAGCCGA | |
| | TGCAAGCGGAGCCGGACCGCGGCACCAAGGCGCGGAAC | |
| | ACGACGCCAGGCCAAGCAGAAGCTCGCCGGCGGCCAGC | |
| | AAGGTCCACAGCGGCGCGGCCACCAGTGCACTCTATGTT | |
| | GAGAGTGGACCAACGTTGACGCCGAAGGCTCACAATGGC | |
| | TGAGCGCTGTAAGTGGCGTCGCGCGCCAGGTTGCCACAC | |
| | CAGGAACAGACGCAACAACCACGAGCGGCCGTACAAGC | |
| | ATAGAGCCGTGCCTGAATGGTTGATCCAGTAGACGGCCA | |
| | CAAGGCAGGTGGCGGTCGAACCGAATGGCTAGGTCCGGC | |
| | TCACAAGGATAACACGGCTACACCTGAACGCCGCCGCAG | |
| | GCGCAACGACGAAGCCGTAGCAACGCAGAGAGATAACG | |
| | CAACGGCCAGGCAAGAGATACACAATTTCATGTCTTGAC | |
| | GGTCCTCGTGAACTGTGGTTAAC | |

What is claimed is:

1. A method comprising:
contacting:
a polynucleotide comprising a template sequence;
a mixture of ribonucleotide triphosphates, wherein the molar ratio of three species of ribonucleotide triphosphates is 1:1:1 and the molar ratio of one species of ribonucleotide triphosphates to any of the other three species is other than 1:1; and
an RNA polymerase,
to produce a polyribonucleotide transcription product comprising a sequence complementary to the template sequence, wherein the polyribonucleotide transcription product comprises fewer base substitution errors than a polyribonucleotide transcription product produced by contacting the polynucleotide template, the RNA polymerase, and an equimolar mixture of the same ribonucleotide triphosphates.

2. A method according to claim 1, wherein the molar ratio of the one species to any of the other three species is more than 1:1.

3. A method according to claim 1, wherein the molar ratio of the one species to any of the other three species is 2:1 or more than 2:1.

4. A method according to claim 1, wherein the molar ratio of the one species to any of the other three species is less than 1:1.

5. A method according to claim 1, wherein the molar ratio of the one species to any of the other three species is 1:2 or less than 1:2.

6. A method according to claim 1, wherein the one species is rATP.

7. A method according to claim 1, wherein the three species of ribonucleotide triphosphates comprise rGTP, rCTP, and rUTP.

8. A method according to claim 1, wherein the three species of ribonucleotide triphosphates comprise rGTP, rCTP, and ψTP.

9. A method according to claim 1, wherein the three species of ribonucleotide triphosphates comprise rGTP, rCTP, and m1ψTP.

10. A method according to claim 1, wherein the RNA polymerase is T3 RNA polymerase, T7 RNA polymerase, Hi-T7 RNA polymerase, KP34 RNA polymerase, or SP6 RNA polymerase.

11. A method according to claim 1 further comprising enzymatically capping the polyribonucleotide transcription product to produce a capped polyribonucleotide following the contacting.

12. A method according to claim 1, wherein the contacting further comprises contacting the polynucleotide, the mixture, the RNA polymerase and a chemical cap analog to produce a chemically capped polyribonucleotide transcription product.

13. A method according to claim 1 further comprising contacting the polyribonucleotide transcription product and one or more pharmaceutically acceptable additives to produce a pharmaceutical dosage form.

14. A method according to claim 1 further comprising contacting the polyribonucleotide transcription product and one or more additives selected from lipidoids, liposomes, polymers, lipoplexes, peptides, proteins, cells transfected with HCMV RNA vaccines, hyaluronidase, and nanoparticles.

15. A method according to claim 1, wherein the coding sequence encodes a polypeptide having an amino acid sequence and the coding sequence comprises no more than 105% the fewest number of uridines possible to encode the amino acid sequence.

16. A method according to claim 1, wherein the coding sequence encodes a polypeptide having an amino acid sequence and the coding sequence comprises no more than 110% the fewest number of uridines possible to encode the amino acid sequence.

17. A method for reducing base substitution errors in a polyribonucleotide transcription product produced by an RNA polymerase, the method comprising:
contacting:
a polynucleotide having a template sequence;
a composition comprising ribonucleotide triphosphates, wherein the molar ratio of the ribonucleotide triphosphates is proportional to the molar ratio of bases in a sequence complementary to the template sequence; and
the RNA polymerase,
to produce the polyribonucleotide transcription product, wherein the polynucleotide transcription product comprises the complementary sequence and wherein the polyribonucleotide transcription product comprises fewer base substitution errors than a polyribonucleotide transcription product produced by contacting the polynucleotide, the RNA polymerase, and a composition comprising the same ribonucleotide triphosphates but in an equimolar ratio.

18. A method comprising:
contacting:
(a) a polynucleotide comprising a template sequence, wherein the molar ratio of bases in a sequence complementary to the template sequence is: wJ: xK: yL: zM,
wherein
ψ, x, y, and z are each independently numbers from 0-50,
J is adenosine or an adenosine analog,
K is uridine or a uridine analog,
L is guanosine or a guanosine analog, and
M is cytidine or a cytidine analog;
(b) a composition comprising ribonucleotide triphosphates, wherein the molar ratio of the ribonucleotide triphosphates is: w′JTP: x′KTP: y′LTP: z′MTP,
wherein
w′, x′, y′, and z′ are each independently numbers from 0-50 (provided no more than 2 of w′, x′, y′, and z′ can equal 0), optionally up to three of w′, x′, y′, and z′ are equal to one another ±10%,
J is adenosine or an adenosine analog,
K is uridine or a uridine analog,
L is guanosine or a guanosine analog, and
M is cytidine or a cytidine analog; and
(c) an RNA polymerase,
to produce a polyribonucleotide transcription product comprising the sequence complementary to the template sequence,
wherein at least one of w′, x′, y′, and z′ is greater or less than each of the other three of w′, x′, y′, and z′, and
wherein the polyribonucleotide transcription product comprises fewer base substitution errors than a polyribonucleotide transcription product produced by contacting the polynucleotide template, the RNA polymerase, and an equimolar mixture of the same ribonucleotide triphosphates.

19. A method according to claim 18, wherein w′ is greater or less than each of x′, y′, and z′.

20. A method according to claim 18, wherein w′ is at least 1.5× greater than each of x′, y′, and z′.

21. A method according to claim 18, wherein x′ is greater or less than each of w′, y′, and z′.

22. A method according to claim 18, wherein x′ is no more than half of each of w′, y′, and z′.

23. A method according to claim 18, wherein y′ is greater or less than each of w′, x′, and z′.

24. A method according to claim 18, wherein y′ is at least 1.5× greater than each of w′, x′, and z′.

25. A method according to claim 18, wherein z′ is greater or less than each of w′, x′, and y′.

26. A method according to claim 18, wherein z′ is at least 1.5× greater than each of w′, x′, and y′.

27. A method according to claim 18, wherein the RNA polymerase is T3 RNA polymerase, T7 RNA polymerase, Hi-T7 RNA polymerase, KP34 RNA polymerase, or SP6 RNA polymerase.

28. A method according to claim 18, wherein:
$$w = w ' \pm 10 \% , x = x ' \pm 10 \% , y = y ' \pm 10 \% , and / or \\ z = z ' \pm 10 \% ,$$

29. A method according to claim 18, wherein at least one of the base substitution errors is rA→rU.

30. A method according to claim 18, wherein J is uridine, pseudouridine, or N¹-methyl-pseudouridine.

31. A method according to claim 18, wherein w is 25-35, x is 3-15, y is 25-35, z is 25-35, w′ is 25-35, x′ is 3-15, y′ is 25-35, and z′ is 25-35.

32. A method according to claim 31, wherein:
$$w = w ' \pm 10 \% , x = x ' \pm 20 \% , y = y ' \pm 10 \% , and / or \\ z = z ' \pm 10 \% ,$$

33. A method according to claim 18 further comprising contacting the transcription product with a pharmaceutically acceptable additive.

34. A method according to claim 18 further comprising contacting the transcription product with a capping enzyme to form a capped transcription product.

35. A method according to claim 34, wherein the capping enzyme is a Faustovirus capping enzyme or a vaccinia capping enzyme.

36. A method according to claim 34 further comprising contacting the capped transcription product with one or more pharmaceutically acceptable additives to produce a pharmaceutical dosage form.

37. A method according to claim 18, wherein the contacting further comprises contacting the polynucleotide, the composition, the RNA polymerase and a chemical cap analog to produce a chemically capped transcription product.


SEQUENCE LISTING (7 sequences)

SEQ ID NO: 1 - DNA, 1142 bp, synthetic construct
  misc_feature 1..1142: note = Synthetic construct
  misc_feature 1..1142: note = RNA1
  source 1..1142: mol_type = other DNA
taatacgactcactatagggtctagaaataattttgtttaactttagagtacacgagtcaggctacagcatcctctggttcagactacttgattcatgtgtaccctatatgcgaggatatgtgtatcgtagaaattgtcaggcagtaacgttccgcgagttttaatgggcgcgccatgactctaagagtgatatacctcctcggtctcgggcccggggtgtaattagcccagttagacacgatcgcccgacgtatattgttgcttgggtatcgtcgcatgcgaagtattgcccaaggagacacaacaagcaacttatgttgactcccttcgaccattaaaatttgttagaacggacagaaaggatgcgccttataaatgtcctgtgcagtgatgaagcgacctcaaaacgcttcatgatctaaccgactcaccttgccgttccctccgcgccttaaaaccggccggtcttgcgaaaagcgggaaacgagtttacccacggatagcagggaatgttgcggctggctagggagcatgaaggtagatactccacggcttacctttccggggctcaacatctagccacagaccttttcgttaagcccacccccactggatactgaatcatcagggaaccggacccaaccagtttgggctcgtccaagcttcggtctcgtccctaagtgcaaagatatggaaagagcagcataggtatatggattattcttttaccactcgtttcttaccgtaacttacgcaatggatcacgtgccgaggcggcggtacagctgttcgaagggctctgtcgggaacgctaacatccagccggtaaattccaaactagggaaaggacacgcactgaattgaatatagtcgtgaagggtggtgtaagtcgtgcacagcccgcattaagtactaaacagcgtccaatcttgatctacttacggcctgatgttcttcagcacctcctagcactggagtacttcgctatcaatgagattagcactttgtacatgtcatccagcccgagtctggggtccgacaatgcggtcgccgattggtatctgcatgtagtattaaacggagctgccgcggctgcggattatagttcatgtcttgacggtcctcgtgaactgtggttaac

SEQ ID NO: 2 - DNA, 1726 bp, synthetic construct
  misc_feature 1..1726: note = Synthetic construct
  misc_feature 1..1726: note = RNA2
  source 1..1726: mol_type = other DNA
taatacgactcactatagggagacccaagcttggtaccgagctcggatccgccaccatgaagaccttaattcttgccgttgcattagtctactgcgccactgttcattgccaggactgtccttacgaacctgatccaccaaacacagttccaacttcctgtgaagctaaagaaggagaatgtattgatagcagctgtggcacctgcacgagagacatactatcagatggactgtgtgaaaataaaccaggaaaaacatgttgccgaatgtgtcagtatgtaattgaatgcagagtagaggccgcaggatggtttagaacattctatggaaagagattccagttccaggaacctggtacatacgtgttgggtcaaggaaccaagggcggcgactggaaggtgtccatcaccctggagaacctggatggaaccaagggggctgtgctgaccaagacaagactggaagtggctggagacatcattgacatcgctcaagctactgagaatcccatcactgtaaacggtggagctgaccctatcatcgccaacccgtacaccatcggcgaggtcaccatcgctgttgttgagatgccaggcttcaacatcaccgtcattgagttcttcaaactgatcgtgatcgacatcctcggaggaagatctgtaagaatcgccccagacacagcaaacaaaggaatgatctctggcctctgtggagatcttaaaatgatggaagatacagacttcacttcagatccagaacaactcgctattcagcctaagatcaaccaggagtttgacggttgtccactctatggaaatcctgatgacgttgcatactgcaaaggtcttctggagccgtacaaggacagctgccgcaaccccatcaacttctactactacaccatctcctgcgccttcgcccgctgtatgggtggagacgagcgagcctcacacgtgctgcttgactacagggagacgtgcgctgctcccgaaactagaggaacctgcgttttgtctggacatactttctacgatacatttgacaaagcaagataccaattccagggtccctgcaaggagattcttatggccgccgactgtttctggaacacttgggatgtgaaggtttcacacaggaatgttgactcttacactgaagtagagaaagtacgaatcaggaaacaatcgactgtagtagaactcattgttgatggaaaacagattctggttggaggagaagccgtgtccgtcccgtacagctctcagaacacttccatctactggcaagatggtgacatactgactacagccatcctacctgaagctctggtggtcaagttcaacttcaagcaactgctcgtcgtacatattagagatccattcgatggtaagacttgcggtatttgcggtaactacaaccaggatttcagtgatgattcttttgatgctgaaggagcctgtgatctgacccccaacccaccgggatgcaccgaagaacagaaacctgaagctgaacgactctgcaatagtctcttcgccggtcaaagtgatcttgatcagaaatgtaacgtgtgccacaagcctgaccgtgtcgaacgatgcatgtacgagtattgcctgaggggacaacagggtttctgtgaccacgcatgggagttcaagaaagaatgctacataaagcatggagacaccctagaagtaccagatgaatgcaaataggcggccgc

SEQ ID NO: 3 - DNA, 1144 bp, synthetic construct
  misc_feature 1..1144: note = Synthetic construct
  misc_feature 1..1144: note = RNA5
  source 1..1144: mol_type = other DNA
taatacgactcactatagggtctagaaataattttgtttaactttagagtacacgagtcaggctacagcatcttgacaccagaatattatggattggacgcttcccactaaatggaagactgttcggtcataaacactactaggaattcctctccagtcatcatgttcgatcgtctagcagcaatctcttccgatcgatatttgcgcgtgactcaggcgagcccatgacagcttctccccgtgagaaccacgactagaagttatctgttgagctgctagcttcgtggcccggccatggtagtagcggctcactcgcgctaactttgcctgctcgagaaaacgggcgaaacacccagcaacacaagccacttaatttgttgatagataataagatcaggttattagtcgctctgcacttactttaagtgccaactatgctgtatcggccagggtgaaaacgggtgccgccacttcagtgtgtcggagtctgctgacggattagggcacagacgtatggttatatcctaaggtagtgtgtcaatgtactggggacaaagtcagtgggcaccgcatcaggagtgcaacctccgctagtaccgactcgtcaatgctttgagcgatggcttgcgctcccaaatccttaagcttttatgcattcggctctggccctcaggcctgacctggaatttcatcggaaacgccttaaccgacattacatcgacaccaagatcccgacgcttcatgcggagacgatagagactctaaccaagaataaaaggagtagtccctaatctactgaaacggggatacctcaaatcacgggaatgcgttactgacccgctatgtgaggctcggatcaccctcgttctattgccttgtaatcatggtggggcggcggagcgggattagagggtgtccctaatgtgagtagatctgtagtaatgatacgtctcctcaatatgaggcgtattgcaggtcacagcacagggagatttcggcgcacccagccgagttgcctccgtcgttgtttaggtatatgcataactgctcacgacaaatacagcagagcctacgttgggttatcgaatccttgtggacaagaagcttcttcatgtcttgacggtcctcgtgaactgtggttaac

SEQ ID NO: 4 - DNA, 4205 bp, synthetic construct
  misc_feature 1..4205: note = Synthetic construct
  misc_feature 1..4205: note = RNA6
  source 1..4205: mol_type = other DNA
taatacgactcactataggagaataaactagtattcttctggtccccacagactcagagagaacccgccaccatgttcgtgttcctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggaatctgcgccagctaccagacacagacaaacagccctcggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatcctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggacctagcgcggccggctagc

SEQ ID NO: 5 - DNA, 1032 bp, synthetic construct
  misc_feature 1..1032: note = Synthetic construct
  misc_feature 1..1032: note = RNA7
  source 1..1032: mol_type = other DNA
taatacgactcactatagggtctagagcgtcggcaagccacctagaacaaccacggcacgcgaaccaacagcggcaacgccgaacggccacgcgacgaactgagcaccaccacaagaccaagccaacgaggaacaccgaacaaccggtcactgaacgcaacggcctaggcgcgcacaccaataagacggatcaagccagcgcatgactgcgccggacaacgccacggacgacgaacaggtccaccggagtacgactatgccataccagcaggacagcggaggcgaccaacaacaacaggagcggcagccggcagacgaattggaccgaccggacatggccacatgcgattgagccggaaccgcgcgcacttgacagtcagcaaccggctccaacagtccggcaccatgagagaaccggcaagccggccttgccacaggaacgcagagagcggagcaacacaagccgacgccaagccaaccgcgaaccagccaggcgccgccggcaacaggaaggtcgcacgagccaacatatggaccggccggaacactaaggaccaagaggccactggcgcggaacggagaaccaggcaccaagcaaggaacagagaggcataggaaccgaaggcgccatagccggcgagacgaccaggaacgaactgacagccaacgctagaggaacgtgacgatggacgaacgtagacacggcggcgcaacgccgcggcggagtccggaaccggcgctaaggtccacacggcagaaccggccaggccgaggcaaccgccgccacacgaccacagcagccagaaggatagcgcgcggccataacaggaacagcaaggacaggcgccacgcaggaccgcaactgagcaaccggcaaccacctccaccgaagccgcgaagcagaggccgacgacggcacacgcgccatgagcggcacaaccagccagaaggccaacgcgccaaggcgaccgaagccaactacagaagtgtccaagccaacgaagccaaccggcagaagagagcagggttaac

SEQ ID NO: 6 - DNA, 1111 bp, synthetic construct
  misc_feature 1..1111: note = Synthetic construct
  misc_feature 1..1111: note = RNA8
  source 1..1111: mol_type = other DNA
taatacgactcactatagggtctagaaataattttgtttaactttagagtacacgagtcaggctacagcatcctccaggcagccacacaacggaggcaagagcggcaagcgcaacaacgcgctccgcgacgagcgcgcagagtatcggcagattccaccggacggagcacgaggacgaagcgaccggtccacacggctggaggacgcagcgccaagctcggtgccgaccgaagccgagcgagaggaacgccgagaagcaagccgcgactccacagagccgccagccgaagaagacgcacgccaggaggaacaggaagcgagcggaaggtcaacgcaccaaggccagcaggactaagccggcagacggcggccgccaccaccaagcgaacaatgatacacgagaacggaccggcaacatggacacggcgtagtccacagagccacaaggcagtggtgcgaaccggcaacggagaacaataccggaagagcacaacagagaggagctggccggcacacgagcgcgacgcggcaccggcgcgccacaccagccgaaggcagccagcaccaacgagcgcggcgcaccgactgacggcgagaacagaccgaaggccggcggccaccacggacgcggaccagaagaccggatggcaggcgacaacgacggctcgcctccaacaagaagcggcggaccggcggaagcgacaggcgccgcacgtgaacacaacaactgcacggcatgcggcgaacggccaagcgagacgacgcgctccaagagccaacgatagaatgctacggcaaccgagcagaggcagagcgccagcgcaacacaccagaagaacgcgacgagagagacggacgccggcgcggaccattggaggcggagaaccgacaaccgcaatgcggagatcaaggcggaccgaacgacaacagcagagccaccaactgcgccgtggtcgcaaccaacctaaccagcaacaacaggcaccgccgcggcgacacaggacgaggccgccgaacaacgataacgcgacaccaggcacacacacgaaccgcggaaggaccacagcgacagccattcatgtcttgacggtcctcgtgaactgtggttaac

SEQ ID NO: 7 - DNA, 1111 bp, synthetic construct
  misc_feature 1..1111: note = Synthetic construct
  misc_feature 1..1111: note = RNA9
  source 1..1111: mol_type = other DNA
taatacgactcactatagggtctagaaataattttgtttaactttagagtacacgagtcaggctacagcatcctcacacaccaatgatccaataacctcgaaggcgacaccgcatgtcgccaccagccgtgaacgacggaagaataaccgcagcgaatacggacatcaagaggcacagaacctacgaagcaacacgaacgcacaccataacttcagcggcaacgaagccgactgaggtccggccggcaaccaaggccgaagttcactaagcctcgtaacggcagccgtataggacctcgccgagcaccagacgacaccaagagaaggcagcgaccaactgcgtcggccggcgaactgccgaagctaaggcacagaccggtcgccaggccgcagaccgcggaaccaacaagaggcagcgctaggccatctcagcaccaccaagacattgatattaacagaacgcggaatcaagcgtacgataccaacgacaagtgtgacgaacgtgcgtctgaatggcacataaggtcgccggtagcgaacgccgaaccgaaccacctaccgcaaccaaccgacaccataggatgtcacagacgactcgcaacggctagaccttaccaacacaggaacatcacaagaatcataacacgagccacaagaagccgatgcaagcggagccggaccgcggcaccaaggcgcggaacacgacgccaggccaagcagaagctcgccggcggccagcaaggtccacagcggcgcggccaccagtgcactctatgttgagagtggaccaacgttgacgccgaaggctcacaatggctgagcgctgtaagtggcgtcgcgcgccaggttgccacaccaggaacagacgcaacaaccacgagcggccgtacaagcatagagccgtgcctgaatggttgatccagtagacggccacaaggcaggtggcggtcgaaccgaatggctaggtccggctcacaaggataacacggctacacctgaacgccgccgcaggcgcaacgacgaagccgtagcaacgcagagagataacgcaacggccaggcaagagatacacaatttcatgtcttgacggtcctcgtgaactgtggttaac